(19) United States
(12) Patent Application Publication
(10) Pub. No.: US 2006/0247439 A1
(43) Pub. Date: Nov. 2, 2006

(54) Mchir antagonists

(75) Inventors: Asim Kumar Ray, Molndal (SE); Emma Margareta Evertsson, Molndal (SE); Anna Stina Maria Linusson Jonsson, Molndal (SE); Pernilla Marie Sandberg, Molndal (SE); Tord Ingiiardt, Molndal (SE); Anette Marie Svensson, Molndal (SE); Kay Brickmann, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, SODERTALJE (SE)

(21) Appl. No.: 10/520,372

(22) PCT Filed: Jul. 4, 2003
(86) PCT No.: PCT/GB03/02884

§ 371 Date: Jan. 9, 2006

(30) Foreign Application Priority Data
Jul. 8, 2002 (SE) 0202134-3

(51) Int. Cl. A61K 31/4709; A61K 31/4706; C07D 409/02; C07D 405/02; C07D 401/02

(52) U.S. Cl. 546/159; 514/313

(57) ABSTRACT

The present invention provides compounds of formula (I), wherein R¹ represents a C₁₋₄ alkoxy group optionally substituted by one or more fluoro or a C₁₋₄alkyl group optionally substituted by one or more fluoro; n represents 0 or 1; R² represents a C₁₋₄alkyl group optionally substituted by one or more fluoro or a C₁₋₄alkoxy group optionally substituted by one or more fluoro; m represents 0 or 1; R³ represents H or a C₁₋₄alkyl group; L¹ represents an alkylene chain (CH₂)ᵣ in which r represents 2 or 3 or L¹ represents a cyclohexyl group wherein the two nitrogens bearing R³ and R⁴, respectively, are linked to the cyclohexyl group either via the 1,3 or the 1,4 positions of the cyclohexyl group or L¹ represents a cyclopentyl group wherein the two nitrogens bearing R³ and R⁴, respectively, are linked to the cyclopentyl group via the 1,3 position of the cyclopentyl group and additionally when R⁵ represents 9,10-methanoanthracen-9(10H)-yl the group -L¹-N(R⁴)— together represents a piperidyl ring which is linked to L² through the piperidinyl nitrogen and to N—R³ via the 4 position of the piperidyl ring with the proviso that when R⁵ represents 9,10-methanoanthracen-9(10H)-yl then r is only 2; R⁴ represents H or a C₁₋₄alkyl group optionally substituted by one or more of the following: an aryl group or a heteroaryl group; L² represents a bond or an alkylene chain (CH₂), in which s represents 1, 2 or 3 wherein the alkylene chain is optionally substituted by one or more of the following: a C₁₋₄alkyl group, phenyl or heteroaryl; R⁵ represents aryl, a heterocyclic group or a CH₃₋₈cycloalkyl group which is optionally fused to a phenyl or to a heteroaryl group; as well as optical isomers and racemates thereof as well as pharmaceutically acceptable salts, thereof; with provisos, processes for preparing such compounds, their use in the treatment of obesity, psychiatric disorders, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders and to pharmaceutical compositions containing them.

### FIELD OF INVENTION

The present invention relates to certain N-cycloalkyl, aryl or heteroaryl N′-quinolin-2-yl alkyldiamines of formula I, to processes for preparing such compounds, to their use in the treatment of obesity, psychiatric and neurological disorders, and to pharmaceutical compositions containing them

### BACKGROUND OF THE INVENTION

Melanin concentrating hormone (MCH) is a cyclic peptide that was first isolated from fish over 15 years ago. In mammals, MCH gene expression is localised to the ventral aspect of the, zona inserta and the lateral hypothalamic area (Breton et al, Molecular and Cellular Neurosciences, vol. 4, 271-284 (1993)). The latter region of the brain is associated with the is control of behaviours such as eating and drinking, with arousal and with motor activity (Baker, B., Trends Endocrinol. Metab. 5: 120-126(1994), vol. 5, No. 3, 120126 (1994)). Although the biological activity in mammals has not been fully defined, recent work has indicated that MCH promotes eating and weight gain (U.S. Pat. No. 5,849,708). Thus, MCH and its agonists have been proposed as treatments for anorexia nervosa and weight loss due to AIDS, renal disease, or chemotherapy. Similarly, antagonists of MCH can be used as a treatment for obesity and other disorders characterised by compulsive eating and excessive body weight. MCH projections are found throughout the brain, including the spinal cord, an area important in processing nociception, indicates that agents acting through MCH1r, such as compounds of formula I, will be useful in treating pain.

Two receptors for MCH (MCH1r (Shimomura et al. Biochem Biophys Res Commun Aug. 11, 1999; 261(3):622-6) & MCH2r (Hilol et al. J Biol Chem Jun. 8, 2001;276(23):20125-9) have been identified in human&, while only one (MCH1r) is present in rodent species (Tan et al Genomics. June 2002;79(6):785-92). In mice lacking MCH1r, here is no increased feeding response to MCH, and a lean phenotype is seen, suggesting that this receptor is responsible for mediating the feeding effect of MCH (Marsh et al. Proc Natl Acad Sci U.S.A. Mar. 5, 2002;99(5):3240-5). In addition, MCH receptor antagonists have been demonstrated to block the feeding effects of MCH (Takekawa et al. Eur J Pharmacol Mar. 8, 2002; 438(3):129-35), and to reduce body weight & adiposity in diet-induced obese rats (Borowsky et al. Nat Med. August 2002;8(8):825-30). The conservation of distribution and sequence of MCH1r suggest a similar role for this receptor in man and rodent species. Hence, MCH receptor antagonists have been proposed as a treatment for obesity and other disorders characterized by excessive eating and body weight.

U.S. Pat. No. 3,020,283 discloses that certain N,N′-bis lepid-2-yl 1,x-diamino C₁₋ₓ alkanes where x is an integer from2 to 12 and N,N′-bis lepid-2-yldiaminocycloalkanes are useful as anthelmintics.

U.S. Pat. No. 5,093,333 discloses certain N-substituted (cyclicaminoalkyl) 2-aminoquinolines which as are useful for treating hypofunction of the cholinergic system and therefore useful in treating dementias involving the cholinergic system.

U.S. Pat. No. 4,203,988 discloses certain pyridinyl and quinolinyl ureas which are useful in treating gastric secretion.

WO99155677 discloses 2-(aminoalkylamino)quinolin-4ones which are useful as anti-bacterial agents.

WO02158702 discloses substituted 2-(aminoalkyl ammo) quinolines which are antagonists of urotensin II which are alleged to be useful in treating cardiovascular diseases characterised by excessive or abnormal vasoconstriction and myocardial dysfunction and also in diseases of the CNS for example addiction, schizophrenia, anxiety and depression and metabolic diseases such as diabetes.

The present invention provides compounds that are MCH1r antagonists which are useful in treating obesity and related disorders, psychiatric disorders, neurological disorders and pain.

### DESCRIPTION OF THE INVENTION

The invention relates to compounds of the general formula (I) 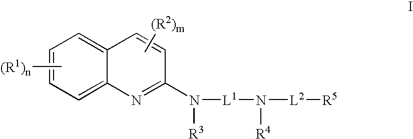
wherein

R¹ represents a C₁₋₄alkoxy group optionally substituted by one or more fluoro or a C₁₋₄alkyl group optionally substituted by one or more fluoro;

n represents 0 or 1;

R² represents a C₁₋₄alkyl group optionally substituted by one or more fluoro or a C₁₋₄alkoxy group optionally substituted by one or more fluoro;

m represents 0 or 1;

R³ represents H or a C₁₋₄alkyl group;

L¹ represents an alkylene chain (CH₂)ᵣ in which r represents 2 or 3 or L¹ represents a cyclohexyl group wherein the two nitrogens bearing R³ and R⁴, respectively, are linked to the cyclohexyl group either via the 1,3 or the 1,4 positions of the cyclohexyl group or L¹ represents a cyclopentyl group wherein the two nitrogens bearing R³ and R⁴, respectively, are linked to the cyclopentyl group via the 1,3 position of the cyclopentyl group and additionally when R⁵ represents 9,10methanoanthracen-9(10H)-yl the group -L¹-N(R⁴)— together represents a piperidyl ring which is linked to L² through the piperidinyl nitrogen and to N—R³ via the 4 position of the piperidyl ring with the proviso that when R⁵ represents 9,10-methanoanthracen-9(10H)-yl then r is only 2;

R⁴ represents H or a C₁₋₄alkyl group optionally substituted by one or more of the following: an aryl group or a heteroaryl group;

L² represents a bond or an alkylene chain (CH₂), in which s represents 1, 2 or 3 wherein the alkylene chain is optionally substituted by one or more of the following: a C₁₋₄alkyl group, phenyl or heteroaryl;

R⁵ represents aryl, a heterocyclic group or a C₃₋₈-cycloalkyl group which is optionally fused to a phenyl or to a heteroaryl group;

as well as optical isomers and racemates thereof as well as pharmaceutically acceptable salts, thereof;

with a first proviso that when n is 0, and m is 1 and R² is methyl located at the 4-position of the quinoline ring, and R³ is H and R⁴ is H and L¹ is (CH₂)₂ or (CH₂)₃ or 1,4cyclohexyl and L² is a bond then R⁵ is not 4-methylquinolin-2-yl;

and with a second proviso that when n is 0, and m is 0 or 1 and R² is a C₁₋₃alkoxy group located at the 4position of the quinoline ring, and R³ is H or a C₁₋₃alkyl group and R⁴ is H or a C₁₋₃alkyl group and L¹ is (CH₂)3 and L² is methylene optionally substituted by one or more C₁₋₃alkyl groups or phenyl then R⁵ is not phenyl, thienyl or indolyl optionally substituted by one, two or three C₁₋₄alkyl groups or-halo.

The term “aryl” as used herein means phenyl naphthyl or 9,10-methanoanthracen-9(10H)-yl, each of which is optionally substituted by one or more of the following: halo, a C₁₋₄alkyl group, phenyl or a group of formula NR⁶R⁷ wherein R⁶ and R⁷ are independently selected from H or a C₁₋₄alkyl group.

The term “heteroaryl” as used herein means thienyl, furyl or pyrrolyl.

The term “heterocyclic group” as used herein means thienyl, furyl, pyridyl, pyrrolyl, quinolinyl indolyl, benzofuranyl or benzo[b]thienyl each of which is optionally substituted by one or more of the following: halo, a C₁₋₄alkyl group, a C₁₋₄acyl group or nitro. In one group of compounds the term “heterocyclic group” means thienyl, furyl pyrrolyl, quinolinyl, indolyl or benzo[b]thienyl each of which is optionally substituted by one or more of the following: halo, a C₁₋₄alkyl group, a C₁₋₄acyl group or nitro.

In one group of compounds of formula (I): R¹ represents a C₁₋₄alkoxy group; n represents 0 or 1; R² represents a C₁₋₄alkyl group; m represents 0 or 1; R³ represents H or a C₁₋₄alkyl group; L¹ represents an alkylene chain (CH₂)ᵣ in which r represents 2 or 3 with the proviso that r is only 2 when R⁵ represents 9,10-methanoanthracen-9(10H)-yl, or L¹ represents a cyclohexyl group wherein the two nitrogens bearing R³ and R⁴, respectively, are inked to the cyclohexyl group either via the 1,3 or the 1,4 positions of the cyclohexyl group and additionally when R⁵ represents 9,10-methanoanthracen-9(10H)-yl the group -L¹-N(R⁴)— together represents a piperidyl ring which is linked to L² through the piperidinyl nitrogen and to N—R³ via the 4 position of the piperidyl ring; R⁴ represents H or a C₁₋₄alkyl group optionally substituted by one or more of the following: an aryl group or a heteroaryl group; L² is represents a bond or an alkylene chain (CH₂)ₛ in which s represents 1, 2 or 3 wherein the alkylene chain is optionally substituted by one or more of the following: a C₁₋₄alkyl group, phenyl or heteroaryl; R⁵ represents aryl a heterocyclic group or a C₃₋₈cycloalkyl group which is optionally fused to a phenyl or to a heteroaryl group; as well as optical isomers and racemates thereof as well as pharmaceutically acceptable salts thereof.

Further particular values of R¹, R², R³, R⁴, R⁵, L¹, L², n, m, r and s in compounds of formula I now follow. It will be understood that such values may be used where appropriate with any of the definitions, claims or embodiments defined hereinbefore or hereinafter.

Particularly R¹ represents a C₁₋₄alkoxy group. More particularly R¹ represents methoxy.

Most particularly R¹ represents 6methoxy when n is 1.

Particularly n represents 1.

Particularly R² represents a C₁₋₄alkyl group. More particularly R² represents methyl. Most particularly R² represents 4-methyl when m is 1.

Particularly m represents 1.

Particularly L¹ represents trimethylene, 1,3-cyclopentyl 1,3-cyclohexyl or 1,4cyclohexyl or when R⁵ represents 9,10-methanoanthracen-9(10H)-yl L¹ additionally represents ethylene. In one group of compounds of formula I, L¹ represents trimethylene. In a second group of compounds of formula I, L¹ represents 1,3-cyclohexyl. In a third group of compounds of formula I, L¹ represents 1,4-cyclohexyl. In a fourth group of compounds of formula I, L¹ represents 1,3cyclopentyl

In a particular group of compounds the group -L¹-N(R⁴)— together represents a piperidyl ring which is linked to L² through the piperidinyl nitrogen and to N—R³ via the 4 position of the piperidyl ring with the proviso that R⁵ represents 9,10-methanoanthracen-9(10H)-yl.

Particularly R³ represents H or a C₁₋₄alkyl group especially methyl. In a particular group of compounds of formula I, R³ represents H.

Particularly L² represents a bond, methylene, methylmethylene, dimethylene optionally substituted by phenyl, or trimethylene optionally substituted by methyl. In a particular group of compounds of formula I, L² is methylene.

Particularly R⁴ represents H or a C₁₋₄alkyl group optionally substituted by a heteroaryl group. More particularly R⁴ represents H, a C₁₋₄alkyl group or thienylmethyl. In a particular group of compounds of formula I, R⁴ represents H. Particularly R⁵ represents phenyl, 2-naphthyl or 9,10-methanoanthracen-9(10H)-yl, each of which is optionally substituted by one or more of the following: methyl, chloro, dimethylamino or phenyl.

More particularly R⁵ represents 4,5,6,7-tetrahydrothianaphth-yl, benzo[b]thien-3-yl, 2-thienyl, 3-thienyl, 2-furyl, 3-furyl, benzofuranyl, pyridyl, 1H-pyrrol-2-yl, 1H-indol-3-yl, or 2-quinolinyl, each of which is optionally substituted by one or more of the following: nitro, methyl, acetyl or chloro.

Most particularly R⁵ represents cyclopropyl, phenyl, 2,4,6-trimethylphenyl, 3,4-dichlorophenyl, 2-naphthyl, 9,10methanoanthracen-9(10H)-yl, 2-thienyl, 3-thienyl, 5-nitro-3-thienyl, 2,5-dimethyl-3-thienyl, 3-furanyl, 5-methyl-2-furanyl, 1-acetyl-1-indol-3-yl, 4,5,6,7-tetrahydrothianaphth-4-yl, benzo[b]thien-3-yl, 1H-indol-3-yl, 2-quinolinyl, 1,1′-biphenyl-4-yl, 4(dimethylamino)phenyl, 1H-pyrrol-2-yl or 2,5-dichloro-3-thienyl.

The term “pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid; or, for example a salt of a compound of Formula I which is sufficiently acidic, for example an alkali or alkaline earth metal salt such as a sodium, calcium or magnesium salt, or an ammonium salt, or a salt with an organic base such as methylamine, dimethylamide, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof. Isomers may be separated using conventional techniques, e.g. chromatography or fractional cystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause raceminsation or epimerisation, or by derivatisation, with a chiral reagent. All stereoisomers are included within the scope of the invention.

The following definitions shall apply throughout the specification and the appended claims.

Unless otherwise stated or indicated, the term “alkyl” denotes either a straight or branched alkyl group. Examples of said alkyl include methyl ethyl, n-propyl, isopropyl n-butyl, iso-butyl, sec-butyl and t-butyl. Preferred alkyl groups are methyl ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine.

The present invention provides a compound selected from:

N-(9,10-methanoanthracen-9(10H)-ylmethyl)-N′(2-quinolinyl)-1,2-ethanediamine;

N-(6-methoxy-methyl-2-quinolinyl)-N′-(3thienylmethyl)-1,3-propanediamine;

N-(9,10-methanoanthracen-9(10H)-ylmethyl)-N′-(2-quinolinyl)-1,3-propanediamine;

N-(2-quinolinyl)-N′-(3-thienylmethyl)-1,3-propanediamine;

N-(9,10-methanoanthracen9(10H)ylmethyl)-N′-(2-quinolinyl 1,4cyclohexanediamine;

N-[(1-acetyl-1H-indol-3-yl)methyl-N′-(6methoxy methyl-2-quinolinyl)-1,3-propanediamine;

N-(9,10methanoanthracen-9(10H)-ylmethyl)-N′-(2-quinolinyl)-1,3-cyclohexanediamine;

N-(2-quinolinyl-N-′-[1-(3-thienyl)ethyl]-1.3-propanediamine;

N-(2-quinolinyl)-N′-(3-thienylmethyl)-1,3-cyclohexanediamine;

N-(9,10-methanoanthracen-9(10H)-ylmethyl)-N′-6-methoxy-4-methyl-2-quinolinyl-1,3-propanediamine;

N-(2-quinolinyl)-N′-(4,5,6,7-tetrahydrothianaphth-4yl)-1,3-propanediamine;

N-methyl-N′-(2-quinolinyl)-N-(3-thienylmethyl)-1,3-propanediamine;

N-(2-quinolinyl)-N′,N′-bis(3-thienylmethyl)-1,3-propanediamine;

N-(9,10-methanoanthracen-9(10H)-ylmethyl)-N-methyl-N′-(2-quinolinyl)-1,3-propanediamine;

N-(2-quinolinyl)-N′-[(2,4,6-trimethylphenyl)methyl]-1,3-propanediamine;

N-(2-phenylethyl)-N′-(2-quinolinyl)-1,3-propanediamine;

N-(1-benzo[b]thien-3-ylmethyl)-N′-(2-quinolinyl)-1,3-propanediamine;

N-[(3,4dichlorophenyl)methyl]-N′-(2-quinolinyl)-1,3-cyclohexanediamine;

N-(9,10methanoanthracen-9(10H)-ylmethyl)-N′-methyl-N′-(2-quinolinyl)-1,3-propanediamine;

N-(2-quinolinyl)-N′-(2-thienylmethyl)-1,3-propanediamine;

N-(3-furanylmethyl)-N′-(2-quinolinyl)-1,3-propanediamine;

N-[(3,4-dichlorophenyl)methyl]-N-methyl-N′-(2-quinolinyl)-1,3-propanediamine;

N-[1-(9,10-methanoanthracen-9(10H)-ylmethyl)-4-piperidinyl]-2-quinolinamine;

N-(1H-indol-3-ylmethyl)-N′-(2-quinolinyl)-1,3-propanediamine;

N-(2-naphthalenymethyl)-N′-(2-quinolinyl)-1,3-propanediamine;

N-(2, 2-diphenylmethyl)-N′-(2-quinolinyl)-1,3-propanediamine;

N-(1H-indol-3-ylmethyl)-N′-(6-methoxy-4-methyl-2-quinolinyl)-1,3-propanediamine;

N-[(3,4-dichlorophenyl)methyl-N′-(2-quinolinyl)-1,3-propanediamine;

N-[(3,4-dichlorophenyl)methyl]-N′-(2-quinolinyl)-1,4-cyclohexanediamine;

N,N′-di-(2-quinolinyl)-1,3-propanediamine;

N-(2-quinolinyl)-N′-(2-quinolinylmethyl)-1,3-propanediamine;

N-[(1-acetyl-1H-indol-3-y)methyl]-N (2-quinolinyl)-1,3-propanediamine;

N-(cyclopropylmethyl)-N′-(2-quinolinyl)-1,3-propanediamine;

N-(2-quinolinyl)-N′-(3-thienylmethyl)-1,4-cyclohexanediamine;

N-([1,1′-biphenyl]-4-ylmethyl)-N′-(2-quinolinyl)-1,3-propanediamine;

N-(6methoxy4-methyl-2-quinolinyl)-N′-[3-(5-methyl-2-furanyl)butyl]-1,3-propanediamine;

N-[(4(dimethylamino)phenyl]methyl]-N′-(2-quinolinyl-1,3-propanediamine;

N-(1H-pyrrol-2-ylmethyl)-N′-(2-quinolinyl)-1,3-propanediamine;

N-[3-(5-methyl-2-furanyl)butyl-N′-(2-quinolinyl)-1,3-propanediamine;

N-[(5-nitro-3-thienyl)methyl]-N′-(2-quinolinyl)-1,3-propanediamine;

N-(6methoxy-4-methyl-2-quinolinyl)-N′-[(5-nitro-3-thienyl)methyl]-1,3-propanediamine;

N-(6methoxy-4-methyl-2-quinolinyl-N′-(1H-pyrrol-2-ylmethyl)-1,3-propanediamine;

N-](3,4-dichlorophenyl)methyl]-N′-methyl-N′-2-quinolinyl)-1,3-propanediamine;

N-[1-(2,5-dimethyl-3-thienyl)ethyl]-N′-2-quinolinyl)-1,3-propanediamine;

N-[1-(2,5-Dichloro-thiophen-3-yl)-ethyl]-N′-(2-quinolinyl)-1,3-propanediamine;

N-[(1-acetyl-1H-indol-3-ylmethyl]-N′-quinolin-2-ylcyclohexane-1,3-diamine;

N-(6-methoxy-4-methylquinolin-2-yl-N′-(3-thienylmethyl)cyclopentane-1,3-diamine;N-(6-methoxy-4-methylquinolin-2-yl)-N′-[(1-methyl-1H-indol-3-yl)methyl]cyclopentane-1,3-diamine;

(1S,3S)-N-(6-methoxy-4-methylquinolin-2-yl)-N′-[(1-methyl-1H-indol-3-yl)methyl]cyclopentane-1,3diamine

(1S,3S)-N-(6-methoxy-4-methylquinolin-2-yl)-N′-(3-thienylmethyl)cyclopentane-1,3-diamine

N-[(1-acetyl-1H-indol-3-yl)methyl]-N′-(6-methoxy-4methylquinolin-2-yl)cyclohexane-1,3-diamine;

N-(1H-indol-3-ylmethyl)-N′-(6methoxy-4-methylquinolin-2-yl)cyclohexane-1,3diamine;

N-(6-methoxy-4-methylquinolin-2-yl)-N′-(3-thienylmethyl)cyclohexane-1,3diamine;

N-(6-methoxy-4-methylquinolin-2-yl)-N′[(1-methyl-1H-indol-3-yl)methyl]cyclohexane-1,3-diamine;

N-(1-benzofuran-2-ylmethyl)N′-(6-methoxy-4-methylquinolin-2-yl)cyclohexane-1,3-diamine; N-(6-methoxy methylquinolin-2-yl)-N′-(pyridin-2-ylmethyl)cyclohexane-1,3-diamine and

N-(4-methylquinolin-2yl)-N′-(3-thienylmethyl)cyclohexane-1,3diamine;

as well as pharmaceutically acceptable salts thereof

Methods of Preparation

The compounds of the invention may be prepared as outlined below according to any of the following methods. However, the invention is not limited to these methods, the compounds may also be prepared as described for structurally related compounds in the prior art.

Compounds of formula I may be prepared by reacting a compound of formula II

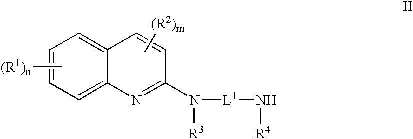

in which R¹, R², R³, R⁴, L¹, n and m are as previously defined with a compound of formula III
R⁵-L^{2′}=O III

in which R⁵ is as previously defied and L^{2′} represents a group which after reaction of compounds II and III gives L² on reduction, under reductive alkylation conditions. For example, a compound of formula II and a compound of formula III may be reacted together at a temperature in the range of 0° C. to 250° C., preferably in the range of 50° C. to 150° C., optionally in the presence of an inert solvent, for example methanol, dichloromethane or acetic acid in the presence of a reducing agent for example (polystyrylmethyl)trimethyl-ammonium cyanoborohydride or sodium cyanoborohydride which is optionally polymer supported

Compounds of formula II may be prepared by reacting a compound of formula IV

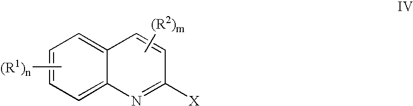

in which R¹, R², n and m are as previously defined and X is halo, particularly chloro or bromo, with a compound of formula V

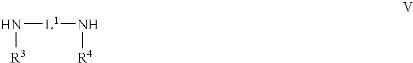

at a temperature in the range of 0° C. to 250° C., preferably in the range of 50° C. to 150° C.,optionally in the presence of an inert solvent, for example toluene, optionally in the presence of a catalytic cross-coupling system for example Pd(OAc)₂ and 2-(di-^{t}butylphosphino)biphenyl or BINAP, and optionally in the presence of a base for example NaO^{t}Bu.

Certain compounds of formula II are novel and are claimed as a further aspect of the present invention as useful intermediates.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction). Optionally a nitrogen in formula V may be protected prior to reaction with a compound of formula IV and then the compound of formula II obtained is deprotected prior to reaction with a compound of formula III. Amine protecting groups are known to those skilled in the art for example the t-BOC group.

The expression “inert solvent” refers to a solvent which does not react with the starting materials, reagents, intermediates or products in a manner which adversely affects the yield of tile desired product.

Pharmaceutical Preparations

The compounds of the invention will normally be administered via the oral, parenteral, intravenous, intramuscular, subcutaneous or in other injectable ways, buccal, rectal, vaginal transdermal and/or nasal route and/or via inhalation, in the form of pharmaceutical preparations comprising the active ingredient either as a free acid, or a pharmaceutically acceptable organic or inorganic base addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

The compounds of the invention may also be combined with other therapeutic agents which are useful in the treatment of disorders associated with obesity, psychiatric disorders, neurological disorders and pain

Pharmacological Properties

The compounds of formula (I) are useful for the treatment of obesity, psychiatric disorders such as psychotic disorders, anxiety, anxio-depressive disorders, depression, cognitive disorders, memory disorders, schizophrenia, epilepsy, and related conditions, and neurological disorders such as dementia, multiple sclerosis, Raynaud's syndrome, Parkinson's disease, Huntington's chorea and Alzheimer's disease. Tie compounds are also potentially useful for the treatment of immune, cardiovascular, reproductive and endocrine disorders, and diseases related to the respiratory and gastrointestinal systems. The compounds are also potentially useful as agents for ceasing consumption of tobacco, treating nicotine dependence and/or treating nicotine withdrawal symptoms, reducing the craving for nicotine and as anti-smoking agents The compounds may also eliminate the increase in weight that normally accompanies the cessation of smoking. The compounds are also potentially useful as agents for treating or preventing diarrhoea.

The compounds are also potentially useful as agents for reducing the craving/relapse for addictive substances that include, but are not limited to psychomotor-active agents such as nicotine, alcohol, cocaine, amphetamines, opiates, benzodiazepines and barbiturates. The compounds are also potentially useful as agents for treating drug addiction and/or drug abuse.

Accordingly, it is desirable to provide a compound and method of trent which will be active in reducing craving for the abused substance, and which does not exacerbate the sympathetic response rate caused by the abused substance and which has favorable pharmacodynamic effects.

The compounds are also potentially useful as agents for treating pain disorders, including but not limited to acute and chronic nociceptive, inflammatory and neuropathic pain and migraine.

In another aspect the present invention provides a compound of formula I as claimed in any previous claim for use as a medicament.

In a further aspect the present invention provides the use of a compound of formula I in the preparation of a medicament for the treatment or prophylaxis of obesity, psychiatric disorders such as psychotic disorders, anxiety, anxio-depressive disorders, depression, bipolar disorder, ADHD, cognitive disorders, memory disorders, schizophrenia, epilepsy, and related conditions, neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of formula I to a patient in need thereof.

In a still further aspect the present invention provides a method of treating obesity, psychiatric disorders such as psychotic disorders, anxiety, anxio-depressive disorders, depression, bipolar disorder, ADHD, cognitive disorders, memory disorders, schizophrenia, epilepsy, and related conditions, and neurological disorders such as dementia, multiple sclerosis, Parkinson's disease, Huntington's chorea and Alzheimer's disease and pain related disorders, including but not limited to acute and chronic nociceptive, inflammatory and neuropathic pain and migraine, comprising administering a pharmacologically effective amount of a compound of formula I to a patient in need thereof.

The compounds of the Sent invention are particulary suitable for the treatment of obesity.

Combination Therapy

The compounds of the invention may be combined with another therapeutic agent that is useful in the treatment of disorders associated with the development and progress of atherosclerosis such as hypertension, hyperlipidaemias, dyslipidaemias, diabetes and obesity. For example, a compound of the present invention may be used in combination with a compound that affects thermogenesis, lipolysis, fat absortion, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of metabolic syndrome or type 2 diabetes and its associated complications, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt, solvate, solvate of such a salt or a prodrug thereof, may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs hereof are well known in the art.

In addition the combination of the invention may be used in conjunction with a sulfonylurea. The present invention also includes a compound of the present invention in combination with a cholesterol-lowering agent The cholesterol-lowering agents referred to in this application include but are not limited to inhibitors of HMG-CoA reductase (3-hydroxy-3-methylglutaryl coenzyme A reductase). Suitably the HMG-CoA reductase. inhibitor is a statin

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (BAT inhibitor). The present invention also includes a compound of the present invention in combination with a bile acid binding resin.

According to au additional further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration one or more of the following agents selected from:

a CETP (cholesteryl ester transfer protein) inhibitor;

a cholesterol absorption antagonist;

a MTP (microsomal transfer protein) inhibitor;

a nicotinic acid derivative, including slow release and combination products; a phytosterol compound;

probucol;

an anti-obesity compound for example orlistat (EP 129,748) and sibutramine (GB 2,184,122 and U.S. Pat. No. 4,929,629);

an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;

a CB1 antagonist or inverse agonist;

another Melanin concentrating hormone (MCH) antagonist;

a PDK inhibitor; or

modulators of nuclear receptors for example LXR, FXR, RXR, and RORalpha;

an SSRI;

a serotonin antagonist;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for for the treatment of type 2 diabetes and its associated complications in a warm-blooded animal, such as man, in need of such treatment which comprises administering to said animal an effective amount of a compound of formula I, or a pharmaceutically acceptable salt, is solvate, solvate of such a salt or a prodrug thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm blooded animal, such as man, in need of such treatment which comprises administering to said animal an effective amount of a compound of formula I, or a pharmaceutically acceptable salt, solvate, solvate of such a salt or a prodrug thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt, solvate, solvate of such a salt or a prodrug thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt, solvate, solvate of such a salt or a prodrug thereof, in a first unit dosage form;

b) a compound from one of the other classes of compounds described in this combination section or a pharmaceutically acceptable salt, solvate, solvate of such a salt or a prodrug thereof; in a second unit dosage form; and

c) container means for containing said first and second dosage forms.

According to a further aspect of the present invention there is provided a kit comprising:

a) a compound of formula I, or a pharmaceutically acceptable salt, solvate, solvate of such a salt or a prodrug thereof, together with a pharmaceutically acceptable diluent or carrier, in a first unit dosage form;

b) a compound from one of the other classes of compounds described in this combination section or a pharmaceutically acceptable salt, solvate, solvate of such a salt or a prodrug thereof, in a second unit dosage form; and

c) container means for containing said first and second dosage forms.

According to another feature of the invention there is provided the use of a compound of the formula I, or a pharmaceutically acceptable salt, solvate, solvate of such a salt or a prodrug thereof, and one of the other compounds described in this combination section, or a pharmaceutically acceptable salt, solvate, solvate of such a salt or a prodrug thereof, in the manufacture of a medicament for use in the the treatment of metabolic syndrome or type 2 diabetes and its associated complications in a warm-blooded animal, such as man.

According to another feature of the invention there is provided the use of a compound of the formula I, or a pharmaceutically acceptable salt, solvate, solvate of such a salt or a prodrug thereof, and one of the other compounds described in this combination section, or s a pharmaceutically acceptable salt, solvate, solvate of such a salt or a prodrug thereof, in the manufacture of a medicament for use in the treatment of hyperlipidaemic conditions in a w blooded animal, such as man.

According to a further aspect of the present invention there is provided a combination treatment comprising the administration of an effective amount of a compound of the formula I, or a pharmaceutically acceptable salt, solvate, solvate of such a salt or a prodrug thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man ill need of such therapeutic treatment.

Working Examples

The invention will now be described in more detail with the following examples that are not to be construed as limiting the invention

### Abbreviations

aq. aqueous
Ac acetyl
BINAP rac-2,2′-Bis(diphenyl-phosphino)-1,1′-binaphtyl
Bu butyl
DMF N,N′-dimethylformamide
EtOAc ethyl acetate
Et₂O diethyl ether
HEK human embryonic kidney
HOAc acetic acid
HPLC high performance liquid chromatography
LC-MS liquid chromatography mass spectroscopy MeOH methanol
Pol-BH₃CN (polystyrylmethyl)trimethylammonium cyanoborohydride
Pol-CHO 4-benzyloxybenzaldehyde polystyrene
TFA trifluoroacetic acid
THF tetrahydrofuran
MeCN acetonitrile
NEt₃ triethylamine
Tris trishydroxymethylaminomethane
t tert
rt. room temperature
sat. saturated
br broad
bs broad singlet
bt broad triplet
d doublet
dd doublet of doublets
m multiplet
q quartet
s singlet
t triplet
tt triplet of triplets
td triplet of doublets
bd broad doublet
   General Experimental Procedures

Flash column chromatography employed Matrex normal phase silica gel 60 Å (30-70) μm. Mass spectra were recorded on a Micromass ZQ single quadrupole equipped with a pneumatically assisted electrospray interface (LC-MS). Purifications were performed on either a semi preparative HPLC with a mass triggered fraction collector, Shimadzu QP 8000, equipped with a XTerra 100 mm×19 mm C18 5 μm column, or on a Waters FractionLynx HPLC with a mass triggered fraction collector, equipped with a Ace μm,5 5 μm C8 100 mm×21.2 mm column or on a Waters Prep LC 2000 with UV-detection, equipped with a Kromasil 10 μm C8 250 mm×20 mm column, or on a semi preparative HPLC, Shimadzu LC-8A,-Shimadzu SPD-10A UV-vis.-detector equipped with a Waters Symmetry® 100 mm×19 mm C18 5 μm column. ¹H NMR and ¹³C NMR spectra were obtained at 298 K on a Varian Unity Plus 400 mHz, or a Varian INOVA 500 MHz or Bruker Avance 300 MHz. Chemical shifts are given in ppm with the solvent residual peak as internal standard: CDCl₃ δ_{H} 7.26, δ_{c} 77.2; MeOH-d₄ δ_{H} 3.31, δhd C 49.0; DMSO-d₆ δ_{H} 2.50; δ_{C} 39.5 ppm, DMF-d₇ δ_{H} 2.75/2.95/8.05, acetone-d₆ δ_{H} 2.05, THF-d₈ δ_{H} 1.74/3.60 ppm. Microwave heating was performed using single node heating in a Smith Creator from Personal Chemistry, Uppsala, Sweden.

Synthesis of Starting Materials and Intermediates

### A1 N-Quinolin-2-ylpropane-1,3-diamine

A mixture of 2-chloroquinoline (4.80 mol 1.0 g), 1,3-propanediamine (7.20 mmol, 0.534 g), NaO^{t}Bu (6.72 mmol, 0.646 g), Pd(OAc)₂ (0.048 mmol, 0.011 g), and 2-(di-^{t}butylphosphino)biphenyl (0.048 mmol, 0.014 g) in toluene (12 mL) was stirred at 100 ° C. under nitrogen until LC-MS indicated that starting material was consumed. The reaction mixture was cooled to room temperature, poured into Et₂O (100 mL) and filtered through a plug of filtration aid. The filtrate was concentrated and the residue purified on a pre-packed SiO₂ column (70 g) eluted with CH₂Cl₂ (containing 0.5% HOAc, 300 mL), CH₂Cl₂:MeOH (5:1,300 mL), and finally with CH₂Cl₂:MeOH:H₂O (10:6:1, containing 1% Bt₃N) to give 0.915 g (95%) of the title compou ¹H NMR (400 MHz, MeOH-d₄) δ 7.85 (d, J=10.1 Hz, 1H), 7.62-7.58. (m, 2H), 7.51 (t, J=8.5 Hz, 1H), 7.20 (t, J =8.0 Hz, 2H), 6.76 (d, J=8.8 Hz, 1H), 3.61 (t, J=6.5 Hz, 2H), 2.92 (t, J=6.6 Hz, 2H),1.93 (quintet, J=6.8 Hz, 2H).

### A2 N-(6-methoxymethyl-2-quinolinyl-1,3-propanediamine

The title compound was prepared from 2-chloro-6-methoxy-4-methylquinoline and 1,3-propanediamine using the procedure described for preparation A1. Yield quantitative. ¹H NMR (400 MHz, DMSO-d₆) δ 7.42 (d, J=9.1 Hz, 1H), 7.12-7.078 (m, 2H), 6.57 (s, 1H), 3.80 (s, 3H), 3.37 (t, J=6.6 Hz, 2H), 2.66 (bt, J=6.6 Hz, 2H), 2.43 (s, 3H), 1.67 (quintet, J=6.8 Hz, 2H).

### A3 N-Quinolin-2-ylcyclohexane-1,4diamine

The title compound was prepared as a mixture of isomers from 2-chloroquinoline and cyclohexane-1,4-diamine using the procedure described for preparation A1. Yield 94%. ¹H NMR (400 MHz, MeOH-₄, major isomer) δ 7.92 (d, J=9.1 Hz, 1H), 7.63 (d, J=8.3 Hz, 1H), 7.60 (d, J=8.1 Hz, 1H), 7.54-7.50 (m, 1H), 7.22 (t, J=8.0 Hz, 1H), 6.92 (d, J=9.3 Hz, 1H), 4.17-4.09 (m, 1H), 3.29-3.21 (m, 1H), 2.22-2.08 (m, 1H), 1.94-1.75 (m, 6H), 1.69-1.37 (m, 1H).

### A4 N-Quinolin-2-ylcyclohexane-1,3-diamine

The title compound was prepared as a mixture of diastereomers from 2-chloroquinoline and cyclohexane-1,3-diamine using tie procedure described for preparation A1. Yield 84%. ¹H NMR (400 Mz, MeOH-d₄, major isomer) δ 7.82 (d, J=8.9 Hz, 1H), 7.61-7.57 (m, 2H), 7.48 (t, J=8.5 Hz, 1H), 7.19 (d, J=7.9 Hz, 1H), 6.73 (d, J=9.1 Hz, 1H), 4.12-4.04 (m, 1H), 3.28-3.21 (m, 2H), 2.56-2.50 (m, 1H), 2.07 (t, J=12.0 Hz, 1H), 1.98-1.93 (m, 1H), 1.82-1.75 (m, 1H), 1.62-1.49 (m, 1H), 1.41-1.23 (m, 2H).

### A5 N-Quinolin-2-ylethane-1, 2-diamine

The title compound was prepared from 2-chloroquinoline and 1,2-ethanediamine using the procedure described for preparation of A1. Yield 65%. ¹H NMR (400 MHz, MeOH-d₈) δ 7.81 (d, J=9.1 Hz, 1H), 7.61-7.56 (m, 2H), 7.47 (t, J=8.5 Hz, 1H), 7.16 (t, J=8.1 Hz, 1H), 6.74 (d, J=8.9 Hz, 1H), 3.55 (t, J=6.2 Hz, 2H), 2.91 (t, J=6.1 Hz, 2H).

### A6 N-Methyl-N′-quinolin-2-ylpropane-1,3diamine

The title compound was prepared from 2-chloroquinoline and N′-methyl-1,3-propanediamine using the procedure described for preparation A1. Yield 61%. ¹H NMR (400 MHz, MeOH-d₄) δ 7.87 (d, J=9.06 Hz, 1H), 7.64-7.59 (m, 2H), 7.56-7.50 (m, 1H), 7.22 (t, J=7.4 Hz, 1H), 6.78 (d, J=8.9Hz, 1H), 3.63 (t, J=6.3 Hz, 2H), 3.03 (t, J=6.5 Hz, 2H), 2.65 (s, 3H), 2.02 (m, 2H).

### A7 N-Methyl-N-quinolin-2-ylpropane-1,3-diamine

The title compound was isolated from preparation A6. ¹H NMR (400 MHz, MeOH-d₄) δ 8.03 (d, J=9.1 Hz, 1H), 7.69-7.59 (m, 211), 7.58-7.52 (m, 1H), 7.22 (t, J=7.4 Hz, 1H), 7.08 (d, J=9.1 Hz, 1H), 3.88 (t, J=6.2 Hz, 2H), 3.16 (s, 3H), 2.94 (t, J=6.4 Hz, 2H), 2.02 (m, 2H).

### A8 N-Piperidin-4-ylquinolin-2-amine

The title compound was prepared from 2-chloroquinoline and piperidin-4-ylamine using the procedure described for preparation A1. Yield 18%. ¹H NMR (400 MHz, MeOH-d₄) δ 7.77 (d, J=9.1 Hz, 1H), 7.59 (d, J=8.3 Hz, 1H), 7.54 (d, J=8.3 Hz, 1H), 7.46 (t, J=8.5 Hz, 1H), 7.21-7.07 (m, 1), 6.71 (d, J=9.8 Hz, 1H), 4.13-4.06 (m, 1H), 3.13 (d, J=12.5 Hz, 2H), 2.80 (dt, J=3.1, 13.7 Hz, 2H), 2.10-2.06 (m, 2H), 1.56-1.46 (m, 2H).

### A9 9-Formyl-9,10-dihydro-9,10-methanoanthracen

Prepared according to literature preparation. H. Sunagawa, et al; Chem. Pharm. Bull. Vol. 27 (1979) pp 1806-1812; U.S. Pat. No. 4,224,344 Sunagawa et al, Sumitomo, Ltd.; Sep. 23, 1980; U.S. Pat. No. 4,358,620 Sunagawa et al, Sumitomo, Ltd; Nov. 9, 1982.

### A10 (1R,3S)-3-[(tert-butoxycarbonyl)amino]cyclopentyl methanesulfonate

Prepared according to literature preparation from (−)-2-azabicyclo[2.2.1]hept-5-en-3-one (>95% ee): H. Bergstrand, et al; Astra A B; New Pharmaceutically Active Compounds; WO9811103; Mar. 19, 1998.

### All tert-butyl [(1S,3S)-3-azidocyclopentyl]carbamate

NaN₃ (16.6 g, 0.25 mmol) was added to a stirred solution of (1R,3S)-3-[(tert-butoxycarbonyl)amino]cyclopentyl methanesulfonate (20 g, crude, ˜0.05 mol) in DMF (250 mL) under nitrogen atmosphere. The mixture was heated to 50° C. for 18 h (over night). The mixture was allowed to reach rt. and poured into H₂O (200 mL) and extracted with EtOAc (2×400 mL), 200 mL Et₂O and concentrated. Purification of the residue by flash chromatography [280 g silica gel, 6×22 cm column, with EtOAc/heptane (2:3→1:1) as eluent) afforded the tide compound (16.5 g, contaminated with DMF) as a slightly yellowish oil taken to the next step without further purification. ¹H NMR (CDCl₃) δ 4.52 (bs, 1H), 4.00-4.10 (m,2H), 1.98-2.22 ([, 3H), 1.62-1.78 (m, 2H), 1.42-1.52 (m, 1H), 1.44 (s, 9 H).

### A12 tert-butyl [(1S,3S)-3-aminocyclopentyl]carbamate

A flask containing tert-butyl [(1S,3S)-3-aminocyclopentyl]carbamate (16.5 g, crude ˜0.05 mol) from A11 and 1.7 g Pd—C (10% paste) in MeOH (300 mL) was exposed to a positive pressure of hydrogen gas (balloon) over weekend The catalyst was filtrated off and the mixture was concentrated to afford the title compound (9.5 g) as a thick colorless viscous oil. ¹H NMR (DMSO-d₆) δ 6,74 (bd, 1H), 3.86-3.92 (m, 1H), 3.28 (quintet, 1H), 1.73-1.98 (m, 2H), 1.43-1.59 (m, 2H), 1.22-1.41 (m, 1H), 1.36 (s, 9 H), 1.07-1.20 (m, 1H), ¹³C NMR (DMSO-d₆) δ 155.0, 77.2, 50.8, 50.0, 42.6, 34.2, 31.2, 28.3. LC-MS [M+H]⁺ 201

### A13 N-(6-methoxy-4-methylquinolin-2-yl)cyclohexane-1,3-diamine

A mixture of 2-chloro-6-methoxy-4methylquinoline (1.20 mmol, 0.250 g), 1,3-cyclohexanediamine (3.61 mmol, 0.412 g), NaO^{t}Bu (1.70 mmol, 0.162 g), Pd(OA)₂ (0.02 mmol 0.004 g), and 2-(di-^{t}butylphosphino)biphenyl (0.034 mmol, 0.010 g) in toluene (5 mL) was stirred at 100° C. under argon for 24 h. The reaction mixture was cooled to room temperature, diluted with EtOAc/MeOH 5:1 containing 1% NEt₃ and loaded directly on a short (˜2cm) silica column. Elution with EtOAc/MeOH 5:1 containing 1% NEt₃ gave 0.241 g (70%) of the title compound as a mixture of diastereorners (˜6:1). ¹H NMR (400 MHz, MeOH-d₄) δ 7.52 (d, J=9.1 Hz, 1H, major isomer), 7.52 (d, J=9.1 Hz, 1H, minor isomer), 7.12 (dd, J=9.1, 2.8 Hz, 1H), 7.05 (d, J=2.8 Hz, 1.H), 6.62 (bs, 1H, minor isomer), 6.53 (bs, 1H, major isomer), 4.27 (m, 1H, minor isomer), 3.88 (tt, J=11.6, 3.8 Hz, 1H, major isomer), 3.80 (s, 3H), 3.02 (m, 1H, minor isomer), 2.76 (tt, J=11.4, 3.8 Hz, 1H, major isomer), 2.44 (bs, 3H, minor isomer), 2.42 (bs, 3H, major isomer), 2.21 (m, 1H), 2.02-0.96 (m, 7H) ; ¹³C NMR (101 MHz, MeOH-d₄, major isomer) δ 156.8, 155.9, 145.3, 144.1, 127.5, 125.1, 120.8, 114.2, 104.8, 55.9, 50.5, 49.6, 43.5, 35.8, 33.6, 24.3, 18.9; LC-MS [M+H]⁺ 286.1.

### A14 N-(4-methylquinolin-2-yl)cyclohexane-1,3-diamine

A solution of 2-chloro-4methylquinoline (0.200 g, 1.13 mmol) and 1,3-diaminocyclohexane (0.51 g, 4.5 mmol) in 3 mL of pyridine was subjected to single node microwave heating (210° C. for 1 h). The reaction mixture was cooled to room temperature and evaporated. The crude product was flash chromatographed on silica gel and eluted with EtOAc/MeOH/Et₃N 50:50:1 to give 0.24 g.(84%) of the title compound as a mixture of diastereomers (˜2.7:1).

¹H NMR (300 MHz, MeOH-d₄) δ 7.7-7.8 (m, 1H), 7.58-7.63 (m, 1H), 7.45-7.55 (m, 1H), 7.18-7.25 (m, 1H), 6.70 (bs, 1H, minor isomer) 6.61 (bs, 1H, major isomer), 4.44 (m, 1H, minor isomer), 4.06 (m, 1H, major isomer), 2.48-2.55 (m, 3H plus 1H, major isomer), 2.32 (m, 1H, minor isomer), 1.2-2.1 (m, 8H).

### EXAMPLES

### Example 1

### N-(9,10-Methanoanthracen-9(10H)-ylmethyl)-N′-(quinolin-2-yl)-1,2-ethanediamine

Pol-BH₃CN (150 mg, pre-swollen in CH₂Cl₂) was added to a solution of N-quinolin-2-ylethane-1,2 diamine (0.299 mmol, 0.056 g) and 9-formyl-9,10-dihydro-9,10-methanoanthracen (0.225 mmol, 0.050 g) in MeOH:CH₂Cl₂ (1:1, containing 1% HOAc, 2.5 mL), and the resultant slurry was subjected to microwave heating single node 100 ° C., 5 min. The resin was filtered off and washed with portions (1-2 mL) of CH₂Cl₂ and MeOH, and the filtrate was concentrate. The residue was dissolved in CH₂Cl₂ (5 mL), and Pol-CHO (140 mg) was added, and the slurry was stirred at room temperature for 60 min. The resin was filtered off and washed with portions (1-2 mL each) of CH₂Cl₂. The filtrate was concentrated, and the residue was purified on SiO₂ (EtOAc:MeOH 9:1) to give 0.078 g (88%) of the title compound. ¹H NMR (400 MHz, MeOH-d₄) δ 7.85 (d, J=8.9 Hz, 1H), 7.56 (dd, J=1.2,9.0 Hz, 1H), 7.39 (dt, J=1.4, 11.5 Hz, 1H), 7.22 (d, J=7.3 Hz, 2H), 7.14 (dt, J=1.2, 7.9 Hz, 1H), 7.12-7.06 (m, 3H), 6.86 (dt, J-1.2, 7.5 Hz, 2H), 6,82-6.75 (m, 3H), 4.30 (s, 1H), 4.02 (s, 2H), 3.80 (t, J=5.2 Hz, 2H), 3.39 (t, J=5.6 Hz, 2H), 2.55 (s, 2H).

Examples 2 to 45 were performed using the procedure described in Example 1 by reacting an amine with an aldehyde as stated.

### Example 2

### N-(6-Methoxy-4-methyl-2-quinolinyl)-N′-(3-thienylmethyl)-1,3-propanediamine

This compound was prepared from N-(6-methoxy-4-methyl-2-quinolinyl)-1,3-propanediamine and 3-thiophenecarboxaldehyde, and purified using HPLC (95% 0.1 M ammonium acetate buffer:5% CH₃CN→100% CH₃CN, 10 min 25 ml/min.) to give the title conpound in 34% yield. ¹H NMR (400 MHz, DMF-d₇) δ 7.48-7.46 (m, 1H), 7.45 (d, J=9.1 Hz, 1H), 7.32-7.31 (m, 1H), 7.17 (dd, J=2.6, 13.5 Hz, 2H), 7.13 (t, J=4.2 Hz, 1H), 6.67 (s, 1H), 3.88 (s, 3H), 3.77 (s, 2H), 3.53 (t, J 6.6 Hz, 2H), 2.69 (t, J=6.7 Hz, 2H), 2.49 (s, 3H), 1.82 (quintet, J=6.7 Hz, 2H).

### Example 3

### N-(9,10-Methanoanthracen-9(10H)-ylmethyl)-N′-(2-quinolinyl)-1,3-propanediamine

This compound was prepared from N-quinolin-2-yl-1,3-propanediamine and 9-formyl-9,10-dihydro-9,10methanoanthracen, and purified on SiO (CH₂Cl₂:MeOH 20:1→10:1, containing 1% HOAc) to give the tide compound in 50% yield. ¹H NMR (MeOH-d₄, 400 MHz) δ 7.85 (d, J=8.9 Hz, 1H), 7.57 (dd, J=1.4, 9.3 Hz, 1H), 7.36-7.32 (m, 5H), 7.31-7.22 (m, 5H), 7.14 (t, J=8.0 Hz, 1H), 7.01-6.93 (m, 5H), 6.75 (d, J=9.1 Hz, 1H), 4.43 (s, 1H), 4.21 (s, 2H), 3.70 (t, J=6.4 Hz, 2H), 3.31 (t, J=1.4 Hz, 2H), 2.65 (s, 2H), 2.23 (quintet, J=6.5 Hz, 2H).

### Example 4

### N-(2-Quinolinyl)-N′-(3-thienylmethyl)-1,3-propanediamine

This compound was prepared from N-quinolin-2-yl-1,3-propanediamine and 3-thiophene-carboxaldehyde, and purified using HPLC (95% 0.1 M ammonium acetate buffer:5% CH₃CN→4 100% CH₃CN, 15 min 25 ml/min.) to give the title compound in 74% yield. ¹H NMR (400 MHz, MeOH-d₄) δ 7.86 (d, J=8.4 Hz, 1H), 7.61 (d, J=8.0 Hz, 1H), 7.50-7.48 (m, 2H), 7.43 (t, J=8.5 Hz, 1H), 7.27 (d, J=8.9 Hz, 1H), 7.20 (t, J=7.7 Hz, 1H), 7.15-7.13 (m, 1H), 6.75 (d, J=9.5 Hz, 1H), 4.23 (s, 2H), 3.65 (t, J=6.2 Hz, 2H), 3.06 (t, J=7.1 Hz, 2H), 2.05 (quintet,J=6.4 Hz, 2H).

### Example 5

### N-(9,10-Methanoanthracen-9(10H)-ylmethyl)-N′-(2-quinolinyl)-1,4-cyclohexanediamine

This compound was prepared from N-quinolin-2-ylcyclohexane-1,4diamine and 9-formyl-9,10-dihydro-9,10-methanoanthracen, and purified using HPLC (95% 0.1 M ammonium acetate buffer.5% CH₃CN→100% CH₃CN, 15 min 25 ml/min.) to give the title conpound as a diastereomeric mixture in 25% yield. ¹H NMR (400 MHz MeOH-d₄, major isomer) δ 7.78 (d, J=9.1 Hz, 1H), 7.59 (d, J=8.5 Hz, 1H), 7.55 (d, J=9.1 Hz, 1H), 7.46 (t, J=8.5 Hz, 1H), 7.24 (d, J=7.7 Hz, 2H), 7.16-7.12 (m, 3H), 6.97-6.89 (m, 4H), 6.79 (d, J=8.9 Hz, 1H), 4.27 (s, 1H), 4.23-4.19 (m, 1H), 3.67 (s, 2H), 2.90-2.85 (m, 1H), 2.51 (d, J=-1.4.Hz, 2H), 1.94-1.85 (m, 4H), 1.82-1.67 (m, 4H).

### Example 6

### N-[(1-Acetyl-1H-indol-3-yl)methyl]-N′-(6-methoxy-4-methyl-2-quinolinyl-1,3-propanediamine

This compound was prepared from N-(6-methoxy-4-methyl-2-quinolinyl)-1,3-propanediamine and 1-acetyl-3-indolecarboxaldehyde, and purified on SiO₂ (CH₂Cl₂:MeOH 40:1→2:1) to give the title compound in 36% yield ¹H NMR (400 MHz, MeOH-d₄, major rotamer) δ 8.33 (d, J=7.5 Hz, 1H), 7.59 (d, J=7.5 Hz, 1H), 7.55 (s, 1H), 7.31. (d, J=7.3 Hz, 1H), 7.26-7.21 (m, 2H), 7.10 (d, J=2.8 Hz, 1H), 6.98 (dd, J=2.8, 11.9 Hz, 1H), 6.54 (s, 1H), 4.08 (s, 2H), 3.84 (s, 3H), 3.57 (t, J=6.3 Hz, 2H), 2.97 (t, J=6.6 Hz, 2H), 2.49 (s, 3H), 2.47 (d, J=0.8 Hz, 3H), 2.01-1.94 (m, 2H).

### Example 7

### N-(9,10-Methanoanthracen-9(10H)-ylmethyl)-N′-(2-quinolinyl)-1,3-cyclohexanediamine

This compound was prepared from N-quinolin-2-ylcyclohexane-1,3-diamine and 9-formyl-9,10-dihydro-9,10-methanoanthracen, and purified using HPLC (95% 0.1 M ammonium acetate buffer:5% CH₃CN→100% CH₃CN, 15 mni 25 ml/min.) to give the title compound as a mixture of diastereomers in 60% yield. ¹H NMR (400 MHz, MeOH-d₄, major isomnr) δ 7.75 (d, J=8.8 Hz, 1H), 7.62 (d, J=8.5 Hz, 1H), 7.53 (d, J=8.6, 1H), 7.46 (dt, 1.2, 7.4 Hz, 1H), 7.23-7.08 (m, 5H), 6.95-6.84 (m, 4H), 6.68 (d, J=9.0 Hz, 1H), 4.23 (s, 1H), 4.15-4.05 (m, 1H), 3.65 (d, J=2.6 Hz, 2H), 2.92-2.81 (m, 1H), 2.53-2.39 (m, 3H), 2.13-2.01 (m, 2H), 1.91-1.81 (m, 2H), 1.60-1.46(m, 1H), 1.29-1.12 (m, 2H).

### Example 8

### N-(2-Quinolinyl)-N′-[1-(3-thienyl)ethyl]-1,3-propanediamine

This compound was prepared from N-quinolin-2-yl-1,3-propanediamine and 3-acetylthiophene, but subjected to microwave heating single node 140° C., 5 min., and purified on SiO₂ (CH₂Cl₂:MeOH 1:0→0:1) to give the title compound in 30% yield. ¹H NMR (400 MHz, MeOH-d₄) δ 7.80 (d, J=9.1 Hz, 1H), 7.58 (d, J=7.9 Hz, 1H), 7.48-7.37 (m, 4H), 7.18 (t, J=7.4 Hz, 1H), 7.06 (d, J=5.0 Hz, 1H), 6.70 (d, J=8.9 Hz, 1H), 4.42-4.38 (m, 1H), 3.59-3.55 (m, 2H), 2.91-2.79 (m, 2H), 2.02-1.93 (m, 2H), 1.56 (d, J=6.7 Hz, 3H).

### Example 9

### N-(2-Quinolinyl)-N′-(3-thienylmethyl)-1,3-cyclohexanediamine

This compound was prepared from N-quinolin-2-ylcyclohexane-1,3diamine and 3-thiophenecarboxaldehyde, and purified using HPLC (95% 0.1 M ammonium acetate buffer.5% CH₃CN→100% CH₃CN, 15 min 25 ml/min.) to give the title compound as a mixture of diastereomers in 33% yield. ¹H NMR (400 MHz, MeOH-d₄, major isomer) δ 7.81 (d, J=8.9 Hz, 1H), 7.58 (t, J=9.1 Hz, 2H), 7.50-7.46 (m, 3H), 7.20-7.15 (m, 2H), 6.71 (d, J=8.9 Hz, 1H), 4.12 (s, 2H), 4.09-4.00 (m, 1H), 3.12-3.04 (m, 1H), 2.50 (d, J=11.9 Hz, 1H), 2.15 (d, J=12.7 Hz, 1H), 2.08 (d, J=14.0 Hz, 1H), 1.98-1.93 (m, 1H), 1.79 (s, 1H), 1.57-1.45 (m, 1H), 1.37-1.21 (m, 2H).

### Example 10

### N-(9,10-Methanoanthracen-9(10H)-ylmethyl)-N′-(6-methoxy4-methyl-2-quinolinyl)-1,3-propanediamine

This compound was prepared from N-(6methoxy-4-methyl-2-quinolinyl)-1,3-propanediamine and 9-formyl-9,1 dihydro-9,10-methanoanthracen, and purified using HPLC (95% 0.1 M ammoniumacetatebuffer:5% CH₃CN→100% CH₃CN, 10 min 25 ml/min.) to give the title compoimd in 20% yield. ¹H NMR (400 MHz, DMF-d₇,) δ 7.36-7.31 (m, 5H), 7.20 (d, J=2.8 Hz, 1H), 7.11 (dd, J=11.9, 2.8 Hz, 1H),6.97 (d, J=3.0 Hz, 2H), 6.95 (d, J=3.2 Hz, 2H), 6.65 (s, 1H), 4.40 (s, 1H), 4.01 (s, 2H), 3.88 (s, 3H), 3.62 (t, J=6.5 Hz, 2H), 3.25-3.21 (m, 2H), 2.61 (s, 2H), 2.49 (s, 3H), 2.14-2.08 (m, 2H).

### Example 11

### N-(2-Quinolinyl)-N′-(4,5,6,7-tetrahydrothianaphth-4-yl)-1,3-propanediamine (alternative name N-quinolin-2-yl-N′-(4,5,6,7-tetrahydro-1-benzothienyl-4-yl)propane-1,3-diamine)

This compound was prepared from N -quinolin-2-yl-1,3-propanediamine and 4-keto-4,5,6,7-tetrahydrothianaphthene, but subjected to microwave heating single node 120° C., 15 mm, and purified on SiO₂ (CH₂Cl₂:MeOH 10:0→4:1) to give the title compound in 34% yield. ¹H NMR (400 MHz, MeOH-d₄) δ 7.82 (d, J=9.3 Hz, 1H), 7.57 (d, J=8.5 Hz, 1H), 7.38 (t, J=8.3 Hz, 1H), 7.22 (d, J=5.7 Hz, 1H), 7.18-7.12 (m, 3H), 6.73 (d, J=8.4 Hz, 1H), 4.19 (t, J=5.9 Hz, 1H), 3.76-3.69 (m, 1H), 3.56-3.50 (m, 1H), 3.00 (t, J=7.2 Hz, 2H), 2.71-2.64 (m, 1H), 2.54-2.47 (m, 1H), 2.09-1.94 (m, 3H), 1.87-1.78 (m, 1H), 1.75-1.65 (m, 1H), 1.64-1.56 (m, 1H).

### Example 12

### N-Methyl-N′-(2-quinolinyl)-N-(3-thienylmethyl)-1,3-propanediamine

This compound was prepared from N-methyl-N′-quinolin-2-ylpropane-1,3-diamine and 3-thiophenecarboxaldehyde, and purified on SiO₂ (CH₂Cl₂:MeOH 10:0→4:1) to give the title compound in 24% yield. ¹H NMR (400 MHz, MeOH-d₄) δ 7.80 (d, J=8.8 Hz, 1H), 7.59-7.55 (m, 2H), 7.46 (dt, J=1.4, 8.0 Hz, 1H), 7.31 (dd, J=2.8, 7.8 Hz, 1H, 7.22 (bs, 1H), 7.16 (dt, J=1.2, 7.4 Hz, 1H), 7.06 (dd, J=1.2.8, 4.7 Hz, 1H), 6.70 (d, J=8.8 Hz, 1H), 3.62 (s, 2H), 3.48 (t, J=6.8 Hz, 2H), 2.54 (t, J=7.3 Hz, 2H), 2.25 (s, 3H), 1.90 (quintet, J=7.0 Hz, 2H).

### Example 13

### N-(2-Quinolinyl)-N′,N′-bis(3-thienylmethyl)-1,3-propanediamine

This compound was prepared from N-quinolin-2-yl-1,3-propanediamine and 3-thiophene-carboxaldehyde, but subjected to microwave beating single node 110° C., 5 min., and purified on SiO₂ (CH₃Cl:MeOH 10:1→2:1) to give the title compound in 30% yield. ¹H NMR (400 MHz, MeOH-d₄) δ 7.82 (d, J=8.8 Hz, 1H), 7.60 (t, J=7.5 Hz, 2H), 7.49 (t, J=8.9 Hz, 1H), 7.32 (m, 1H), 7.23 (bs, 2H), 7.19 (m, 2H), 7.10 (d, J=4.2 Hz, 2H). 6.65(d, J=9.1 Hz, 1H), 3.65 (s, 4H), 3.49 (t, J=6.6 Hz, 2H), 2.59 (t, J=6.6 Hz, 2H), 1.91 (quintet, J=7.0 Hz, 2H).

### Example 14

### N-(9,10-Methanoanthracen-9(10H)-ylmethyl)-N-methyl-N′-(2-quinolinyl)-1,3-propanediamine

This compound was prepared from N-methyl-N′-quinolin-2-ylpropane-1,3-diamine and 9-formyl-9,10-dihydro-9,10-methanoanthracen, and purified on SiO₂ (CH₂Cl₂:MeOH 10:0→4:1) to give the title compound in 11% yield. ¹H NMR (400 MHz, MeOH-d₄) δ 7.71 (d, J=8.8 Hz, 1H), 7.56 (t, J=8.2 Hz, 2H), 7.45 (t, J=7.4 Hz, 1H), 7.19-7.1 (m, 5H), 6.89-6.83 (m,4H), 6.40 (d, J=8.8 Hz, 1H), 4.20 (s, 1H), 3.51-3.48 (m, 4H), 2.76 (t, J=6.9 Hz, 2H), 2.56 (s, 2H), 2.43 (s, 3H), 1.96-1.89 (m, 2H).

### Example 15

### N-(2-Quinolinyl-N′-[(2,4,6-trimethylphenyl)methyl]-1,3-propanediamine

This compound was prepared from N-quinolin-2-yl-1,3-propanediamine and 2,4,6-trimethyl-benzaldehyde, and purified using HPLC (95% 0.1 M ammonium acetate buffer:5% CH₃CN→100% CH₃CN, 15 mm 25 ml/min.) to give the title compound in 27% yield ¹H NMR (400 MHz, MeOH-d₄) δ 7.87 (d, J=9.0 Hz, 1H), 7.59 (dd, J=9.3, 1.6 Hz, 1H), 7.27-7.23 (m, 1H), 7.18-7.14 (m, 1H), 6.96 (s, 2H), 6.90 (d, J=8.4 Hz, 1H), 6.78 (d, J=8.9 Hz, 1H), 4.30 (s, 2H), 3.71 (t, J=6.2 Hz, 2H), 3.21 (t, J=6.7 Hz, 2H), 2.39 (s, 6H), 2.31 (s, 3H), 2.16 (quintet, J 6.5 Hz, 2H).

### Example 16

### N-(2-Phenylethyl)-N′-(2-quinolinyl)-1,3-propanediamine

This compound was prepared from N-quinolin-2-yl-1,3-propanediamine and phenyl acetaldehyde, and purified using HPLC (95% 0.1 M ammonium acetate buffer:5% CH₃CN→100% CH₃CN, 15 nin 25-ml/min) to give the title compound in 4% yield. ¹H NMR (400 MHz, MeOH-d₄) δ 7.88 (d, J=9.0 Hz, 1H), 7.65-7.52.(m, 3H), 7.30-7.19 (m, 4H), 7.15 (d, J=1.7 Hz, 1H), 7.13 (s, 1H), 6.77 (d, J=9.1 Hz, 1H), 3.65 (t, J=6.2 Hz, 2H), 3.22-3.18 (m, 2H), 3.11 (t, J=6.8 Hz, 2H), 2.95-2.91 (m, 2H), 2.04 (quintet, J=6.5 Hz, 2H).

### Example 17

### N-(1-Benzo[b]thien-3-ylmethyl)-N′-(2-quinolinyl)-1,3-propanediamine

This compound was prepared from N-quinolin-2-yl-1,3-propanediamine and 3-acetylthianaphthene but subjected to microwave heating single node 120° C., 2×5 min., and purified on SiO₂ (CH₂Cl₂:MeOH 10:0→4:1) to give the title compound in 30% yield.

¹H NMR (400 MHz, MeOH-d₄) δ 7.88-7.80 (m, 2H), 7.77 (d, J=8.9 Hz, 1H), 7.58 (s, 1H), 7.55 (dd, J=1.4, 9.1 Hz, 1H), 7.37-7.27 (m, 4H), 7.14 (t, J=8.0 Hz, 1H), 6.66 (d, J=9.2 Hz, 1H), 4.70 (q, J=6.9 Hz, 1H), 3.64-3.52 (m, 2H), 3.03-2.97 (m, 1H), 2.91-2.85 (m, 1H), 1.98 (octet, J=6.7 Hz, 2H), 1.65 (d, J=6.6 Hz, 3H).

### Example 18

### N-[(3,4-Dichlorophenyl)methyl]-N′-(2-quinolyl)-1,3-cyclohexanediamine

This compound was prepared from N-quinolin-2-ylcyclohexane-1,4-diamine and 3,4-dichlorobenzaldehyde, and purified using HPLC (95% 0.1 M ainonium acetate buffer:5% CH₃CN→100% CH₃CN, 15 min 25 ml/min.) to give the title compound as a diastereomeric mixture in 66% yield. ¹H NR (400 MHz, MeOH-d₄, major isomer) δ 7.79 (d, J=8.9 Hz, 1H), 7.60-7.53 (m, 3H), 7.50-7.45 (m, 2H), 7.31 (dd, J=2.0, 10.1 Hz, 1H), 7.18-7.14 (m, 1H), 6.70 (d, J=9.2Hz, 1H), 4.04-3.96 (m, 1H), 3.89 (s, 2H), 2.88-2.81. (m, 1H), 2.47 (d, J=12.1 Hz, 1H), 2.06 (d, J=12.1 Hz, 2M, 1.92-1.86 (m, 1H), 1.80-1.67 (m, 1H), 1.54-1.42 (m, 1H), 1.29-1.12 (m, 2H).

### Example 19

### N-(9,10-Methanoanthracen-9(10H)-ylmethyl)-N-methyl-N′-(2-quinolinyl-1,3-propanediamine

The title compound was isolated from synthesis of Example 14. ¹H NMR (400 MHz, MeOH-d₄) δ 7.90 (d, J.=9.0 Hz, 1H), 7.56 (d, J=8.3 Hz, 1H), 7.35 (t, J=8.2 Hz, 1H), 7.27-7.23 (m, 3H), 7.15-7.10 (m, 3H, 7.02 (d, J=8.8 Hz, 1H), 6.94-6.86 (m, 4H), 4.26 (s, 1H), 3.87 (t, J 6.9 Hz, 2H), 3.63 (s, 2H), 3.18 (s,. 3H), 2.85 (t, J=6.6 Hz, 2H), 2.49 (s, 2H), 2.01 (quintet, J=7.0 Hz, 2H).

### Example 20

### N-(2-Quinolinyl)-N′-(thienylmethyl)-1,3-propanediamine

This compound was prepared from N-quinolin-2-yl-1,3-propanediamine and 2-thiophenecarboxaldehyde, and purified using HPLC (95% 0.1 M ammonnium acetate buffer:5% CH₃CN→100% CH₁CN₃, 15 min 25 ml/min.) to give the title compound in 18% yield. ¹H NMR (400 MHz, MeOH-d₄) δ 7.84 (d, J=8.9 Hz, 1H), 7.60 (dd, J=1.7, 9.3 Hz, 1H), 7.47-7.42 (m, 3H), 7.37 (d, J=8.4 Hz, 2H), 7.20-7.17 (m, 1H), 7.10 (d, J=3.2 Hz, 1H), 7.00 (dd, J=3.7, 8.4 Hz, 1H), 6.74 (d, J=9.4 Hz, 1H), 4.28 (s, 2H), 3.61 (t, J=6.5 Hz, 2H), 2.96 (t, J=7.1 Hz, 2H), 2.00 (quintet, J=6.8 Hz, 2H).

### Example 21

### N-(3-Furanylmethyl)-N′-(2-quinolinyl)-1,3-propanediamine

This compound was prepared from N-quinolin-2-yl-1,3-propanediamine and 3-furaldehyde, and purified using HPLC (95% 0.1 M amnmonium acetate buffer:5% CH₃CN→100% CH₃CN, 15 min. 25 ml/min.) to give the title conpound in 21% yield. ¹H NMR (400 MHz, MeOH-d₄) δ 7.86 (d, J=8.4 Hz, 1H), 7.61 (d, J=9.5 Hz, 1H), 7.54 (d, J=6.8 Hz, 21), 7.50-7.41 (m, 2H), 7.21 (t, J=8.1 Hz, 1H), 6.75 (d, J=9.1 Hz, 1H), 6.46 (t, J=0.9 Hz, 1H), 4.04 (s, 2H), 3.64 (t, J=6.4 Hz, 2H), 3.02 (t, J=6.7 Hz, 2H) 2.03 (quintet, J=6.6 Hz, 2H).

### Example 22

### N-[(3,4-Dichlorophenyl)methyl]-N-methyl-N′-(2-quinolinyl)-1,3-propanediamine

This compound was prepared from N-methyl-N′-quinolin-2-ylpropane1,3-diamine and 3,4-dichlorobenzaldehyde, and purified on SiO₂ (CH₂Cl₂:MeOH 10:0→4:1) to give the title compound in 20% yield. ¹H NMR (400 MHz, MeOH-d₄) δ 7.79 (d, J=9.3 Hz, 1H), 7.60-7.55 (m, 2H), 7.49-7.44 (m, 2H), 7.33 (d, J=9.3 Hz, 1H), 7.22-7.13 (m, 2H), 6.68 (d, J=8.8 Hz, 1H), 3.49 (t, J=7.4 Hz, 2H), 3.49 (s, 2H), 2.52 (t, J=7.4 Hz), 2.22 (s, 3H), 1.87 (quintet, J=7.2 Hz, 2H).

### Example 23

### N-[1-(9,10-Methanoanthracen-9(10H)-ylmethyl)-4-piperidinyl]-2quinolinamine

This compound was prepared from N-piperidin-4-ylquinolin-2-amine and 9-formyl-9,10-dihydro-9,10-methanoanthracen, and purified using HPLC (95% 0.1 M ammonium acetate buffer:5% CH₃CN→100% CH₃CN, 15 min 25 ,l/min.) to give the title compound in 53% yield. ¹H NMR (400 MHz, THF-d₈) δ 7.77 (d, J=9.0 Hz, 1H), 7.64 (d, J=9.1 Hz, 1H), 7.57 (d, J=8.2 Hz, 1H), 7.47 (t, J=8.4 Hz, 1H), 7.27 (d, J=6.6 Hz, 4H), 7.15 (t, J=8.0 Hz, 1H), 6.99-6.90 (m, 4H), 6.68 (d, J=9.0 Hz, 1H), 4.30 (s, 1H), 4.22-4.15 (m, 1H), 3.51 (s, 2H), 3.12 (d, J=11.9 Hz, 2H), 2.63 (s, 2H), 2.52 (dt, J=2.6, 12.6 Hz, 2H), 2.14 (d, J=13.2 Hz, 2H), 1.59 (dq, J=4.4, 12.7 Hz, 2H).

### Example 24

### N-(1H-Indol-3-ylmethyl)-N′-(2-quinolinyl)-1,3-propanediamine

This compound was prepared from N-quinolin-2-yl-1,3-propanediamine and indole-3-carboxaldehyde, and purified using HPLC (95% 0.1 M ammonium acetate buffer:5% CH₃CN→100% CH₃CN, 15 min 25 ml/min.) to give the title compound in 19% yield. ¹H NMR (400 MHz, MeOH-d4) δ 7.83 (d, J=8.9 Hz, 1H), 7.63 (d, J=8.6 Hz, 1H), 7.58 (d, J=8.2 Hz, 1H), 7.41 (d, J=8.5 Hz, 1H), 7.33-7.29 (m, 2H), 7.19-7.13 (m, 3H), 7.06 (t, J=7.7 Hz, 1H), 6.72 (d, J=9.4Hz, 1H), 4.41 (s, 2H), 3.66 (t, J=6.1 Hz, 2H), 3.10 (t, J=6.7 Hz, 2H), 2.06 (quintet, J=6.6 Hz, 2H).

### Example 25

### N-(2-Naphthylmethyl)-N′-(2-quinolinyl)-1,3-propanediamine

This compound was prepared from N-quinolin-2-yl-1,3-propanediamine and 2-naphthaldehyde, but the reaction was performed at room temperature (no microwave heating single node) using NaBH₃CN, and purified on SiO₂ (CH₂Cl₂:MeOH 40:1→10:1, containing 1% HOAc) to give the title compound in 73% yield. ¹H NMR (400 MHz, MeOH-d₄) δ 7.91-7.87 (m, 4H), 7.80-7.77 (m, 7.61 (d, J=8.3 Hz, 1H), 7.56-7.50 (m, 3H), 7.27-7.16 (m, 3H), 6.79 (d, J=9.1 Hz, 1H), 4.38 (s, 2H), 3.68 (t, J=6.3 Hz, 2H), 3.18 (t, J=7.2 Hz, 2H), 2.12 (quintet, J=6.6 Hz, 2H).

### Example 26

### N-(2,2-Diphenylethyl)-N′-(2-quinolinyl)-1,3-propanediamine

This compound was prepared from N-quinolin-2-yl-1,3-propanediamine and diphenyl-acetaldehyde, but the reaction was performed at room temperature (no microwave heating single node) using NaBH₃CN, and purified on SiO₂ (CH₂Cl₁:MeOH 30:1→10:1, containing 1% HOAc) to give the title compound in 53% yield. 1H NMR (400 MHz, MeOH-d₄) δ 7.86 (d, J=9.1 Hz, 1H), 7.61 (d, J=7.0 Hz, 1H), 7.45 (t, J=8.3 Hz, 1H), 7.34-7.19 (m, 12H), 6.73 (d, J=8.9 Hz, 1H), 4.32 (t, J=8.0 Hz, 1H), 3.75 (d, J=8.0 Hz, 2H), 3.58 (t, J=6.2 Hz, 2H), 3.08 (t, J=7.2 Hz, 2H), 2.05-1.98 (m, 2H).

### Example 27

### N-(1H-Indol-3-ylmethyl)-N′-(6-methoxy-4-methyl-2-quinolinyl)-1,3-propanediamine

This compound was prepared from N-(6methoxy-4-methyl-2-quinolinyl)-1,3-propanediamine and indole-3-carboxaldehyde, and purified using HPLC (95% 0.1 M ammonium macerate buffer:5% CH₃CN→100% CH₃CN, 15 min. 25 ml/min.) to give the title compound in 22% yield ¹H NMR (400 MHz, MeOH-d₄) δ 7.60 (d, J=8.5 Hz, 1H), 7.41 (d, J=8.2 Hz, 1H), 7.31 (s, 1H), 7.18-7.02 (m, 4H), 6.96 (dd, J=2.7, 12.0 Hz, 6.61 (s, 1H), 4.38 (s, 2H), 3.84 (s, 3), 3.61 (t, J=5.8Hz, 2H), 3.09 (t, J=6.6 Hz, 2H), 2.50 (d, J=0.8 Hz, 3H), 2.04 (quintet, J 6.5 Hz, 2H).

### Example 28

### N-[(3,4-Dichlorophenyl)methyl-N′-(2-quinolinyl)-1,3-propanediamine

This compound was prepared from N-quinolin-2-yl-1,3-propanediamine and 3,4-dichloro-benzaldehyde, and purified using HPLC (95% 0.1 M ammonium acetate buffer.5% CH₃CN→100% CH₃CN, 15 min 25 ml/min.) to give the title compound in 44% yield. ¹H NMR (400 MHz, MeOH-d₄) δ 7.82 (d, J=9.3 Hz, 1H), 7.58 (d, J=8.2 Hz, 1H), 7.50 (d, J=2.2 Hz, 1H), 7.44-7.41 (m, 2H), 7.37 (d, J=8.3 Hz, 1H), 7.24 (dd, J=2.5, 10.5 Hz, 1H), 7.19-7.15 (m, 1H), 6.72 (d, J=8.9 Hz, 1H), 3.92 (s, 2H), 3.59 (t, J=7.5 Hz, 2H), 2.87 (t, J=7.6 Hz, 2H), 1.96 (quintet, J=6.7 Hz, 2H).

### Example 29

### N-[(3,4-Dichlorophenyl)methyl]-N′-(2-quinolinyl)-1,4-cyclohexanediamine

This compound was prepared from N-quinolin-2-ylcyclohexane-1,4diamine and 3,4-dichlorobenzaldehyde, and purified using HPLC (95% 0.1 M ammonium acetate buffer:5% CH₃CN→100% CH₃CN, 15 min. 25 ml/min.) to give the title compound as a mixture of isomners in 45% yield. ¹H NMR (400 MHz, MeOH-d₄) δ 7.84 (d, J=9.1 Hz, 1H), 7.65-7.46 (m, 5H), 7.35 (dd, J=2.0, 10.3 Hz, 1H), 7.20-7.15 (m, 1H), 6.82 (d, J=9.1 Hz, 1H), 4.20-4.16 (m, 1H), 3.95 (s, 2H), 2.92-2.85 (m, 1H), 2.22-2.16 (m, 1H), 2.02-1.98 (m, 2H), 1.89-1.84 (m, 2H), 1.79-1.67 (m, 3H).

### Example 30

### N,N′-Di-(2-quinolinyl)-1,3-propanediamine

The title compound was isolated in 3% yield from synthesis of 2-quinolinyl-1,3-propanediamine. ¹H NMR (400MHz, MeOH-d₄) δ 7.77 (d, J=8.5 Hz, 2H), 7.71 (d, J=8.9 Hz, 2H), 7.55 (m, 4H), 7.20 (t, J=7.8 Hz, 2H), 6.61 (d, J=8.9 Hz, 2H), 3.59 (bs, 4H), 1.92 (bt, J=5.7 Hz, 2H).

### Example 31

### N-(2Quinolinyl-N′-(2-quinolinylmethyl)-1,3-propanediamine

This compound was prepared from N-quinolin-2-yl-1,3-propanediamine and 2-quinoline-carboxaldehyde, and purified on SiO₂ EtOAc:MeOH 1:0→0:1) to give the title compound in 27% yield. ¹H NMR (400 MHz, MeOH-d₄) δ 8.23 (d, J=8.5 Hz, 1H), 7.98 (d, J=8.5 Hz, 1H), 7.88 (d, J=8.1 Hz, 1H), 7.77 (d, J=8.9 Hz, 1H), 7.74-7.70 (m, 1H), 7.58-7.47 (m, 4H), 7.33 (t, J=8.5 Hz, 1H), 7.12 (t, J=8.0 Hz, 1H), 6.69 (d, J=8.7 Hz, 1H), 4.13 (s, 2H), 3.60 (t, J=6.6 Hz, 2H), 2.87 (t, J=6.9 Hz, 2H), 1.96.(quintet, J=6.7 Hz, 2H).

### Example 32

### N-[(1-Acetyl-1H-indol-3-ylmethyl]-N′-quinolinyl-1,3-propanediamine

This compound was prepared from N-quinolin-2-yl-1,3-propanediamine and 1-acetyl-3-indolecarboxaldehyde, and purified using HPLC (95% 0.1 M amnnium acetate buffer:5% CH₃CN→100% CH₃CN, 10 min 25 ml/min.) to give tbh title compoumd in 25% yield ¹H NMR (400MHz, acetone-d₆, major rotamer) δ 7.77 (d, J=8.9 Hz, 1H), 7.69 (d,J=8.7 Hz, 1H), 7.61 (s, 1H), 7.57 (dd, J=9.3, 1.4 Hz, 1H), 7.52 (d, J=8.5 Hz, 1H), 7.38 (d, J=7.5 Hz, 1H), 7.28 (s, 1H), 7.10 (d, J=8.9 Hz, 2H), 7.02-6.98 (m, 1H), 6.69 (d, J=8.9 Hz, 1H), 4.01 (s, 2H), 3.64-3.61 (m, 2H), 2.86 2.81 (m, 2H), 2.53 (s, 3H), 1.90-1.86 (m, 2H).

### Example 33

### N-(Cyclopropylmethyl)-N′-(2-quinolinyl)-1,3-propanediamine

This compound was prepared from N-quinolin-2-yl-1,3-propanediamine and cyclopropanecarboxaldehyde, and purified using HPLC (95% 0.1 M amnium acetate buffer: 5% CH₃CN→100% CH₃CN, 15 min 25 ml/min.) to give the title compound in 17% yield. ¹H NMR (400 MHz, MeOH-d₄) δ 8.07 (d, J=8.1 Hz, 1H), 7.74 (t, J=6.4 Hz, 2H), 7.65 (t, J=7.8 Hz, 1H), 7.36 (t, J=7.5 Hz, 1H), 6.93 (d, J=8.7 Hz, 1H), 3.67 (t, J=6.6 Hz, 2H), 3.16 (t, J=7.3 Hz, 2H), 2.93 (d, J=7.5 Hz, 2H), 2.10 (quintet, J=7.3 Hz, 2H), 1.12-1.02 (m, 1), 0.71-0.67 (m, 2H), 0.40-0.37 (m, 2H).

### Example 34

### N-(2-Quinolinyl)-N′-(3-thienylmethyl)-1,4-cyclohexanediamine

This compound was prepared from N-quinolin-2-ylcyclohexane-1,4-diamine and 3-thiophenecarboxaldehyde, and purified using HPLC (95% 0.1 M aromnium acetate buffer:5% CH₃CN→100% CH₃CN, 15 min 25 ml/min.) to give the title compound as a diastereomeric mixture in 27% yield. ¹H NMR (400 MHz, MeOH-d₄, major isomer) δ 7.78 (d, J=8.9 Hz, 1H), 7.58 (d, J=8.5 Hz, 1, 7.55 (dd, J=9.3, 1.2 Hz, 1H), 7.45 (t, J=8.5 Hz, 1H), 7.35 (dd, J=7.9, 3.0 Hz, 1H), 7.26-7.24 (m, 1H), 7.16-7.10 (m, 2H), 6.78 (d, J=9.1 Hz, 1H), 4.18-4.16 (m, 1H), 3.81 (s, 2H), 2.65 (septet, J=4.1 Hz, 1H), 1.92-1.83 (m, 2H), 1.80-1.64 (m, 5H), 1.64-1.54 (m, 1H).

### Example 35

### N-([1,1′-Biphenyl]-4ylmethyl)-N′-(2-quinolinyl)-1,3-propanediamine

This compound was prepared from N-quinolin-2-yl-1,3-propanediamine and 4-biphenylcarboxaldehyde, but the reaction was performed at room temperature (no microwave heating single node) using NaBH₃CN, and purified on SiO₂ (CH₂Cl₂:MeOH 30:1→10:1, containing 1% HOAc) to give the title compound in 46% yield. ¹H NMR (400 MHz, MeOH-d₄) δ 7.84 (d, J=9.2 Hz, 1H), 7.62-7.⁵⁶(m, 5H), 7.48-7.40 (m, 5H) 7.36 (t, J=7.1 Hz, 1H),7.23 (d, J=8.5 Hz, 1H), 7.16 (t, J=8.5 Hz, 1H), 6.74 (d, J=8.5 Hz, 1H), 4.21 (s, 2H), 3.66 (t, J=5.8 Hz, 2H), 3.08 (t, J=7.0 Hz, 2H), 2.07 (m, 2H).

### Example 36

### N-(6-Methoxy-4-methyl-2-quinolinyl)-N′-[3-(5-methyl-2furanyl)butyl]-1,3-propanediamine

This compound was prepared from N-(6-methoxy-4-methyl-2-quinolinyl)-1,3-propanediamine and 3-(5-methyl-2-furyl)butyraldehyde, and purified using HPLC (95% 0.1 M ammonium acetate buffer:5% CH₃CN→100% CH₃CN, 10 min 25 ml/mm ) to give the title compound in 46% yield. ¹H NMR (400 MHz, MeOH-d₄) δ 7.54 (d, J=8.9 Hz, 1H), 7.22 (dd, J=2.6, 8.7 Hz, 1H), 7.19 (d, J=2.8 MHz, 1H), 6.72 (d, J=1.0 Hz, 5.96-5.94 (m, 2H), 3.92 (s, 3M, 3.56 (t, J=6.6Hz), 2.93-2.88 (m, 2H), 2.77-2.75 (m, 2H), 2.66 (t, J=7.4 Hz, 2H), 2.53 (d,J=0.8 Hz, 3H), 2.25 (d, J=0.8 Hz, 3H), 1.90-1.82 (m, 2H), 1.72-1.67 (m, 1H), 1.21 (d, J=7.0 Hz, 3H).

### Example 37

### N-[[4-(Dimethylamino)phenyl]methyl]-N′-(2quinolinyl)-1,3-propanediamine

This compound was prepared from N-quinolin-2-yl-1,3-propanediamine and 4-dimethyl-aminobenzaldehyde, mad purified using HPLC (95% 0.1 M ammonium acetate buffer:5% CH₃CN→100% CH₃CN, 15 min 25 ml/min.) to give the title compound in 22% yield ¹H NMR (400 MHz, MeOH-d₄) δ 7.85 (d, J=9.0 Hz, 1H), 7.60 (dd, J=1.5, 9.4 Hz, 1H), 7.39 (t, J=8.5 Hz, 1H), 7.24-7.17 (m, 4H), 6.74 (d, J=9.1 Hz, 1H), 6.70 (d, J=9.0 Hz, 2H), 4.07 (s, 2H), 3.65 (t, J=6.2 Hz, 2H), 3.04 (t, J=6.6 Hz, 2H), 2.94 (s, 6H), 2.05 (quintet, J=6.6 Hz, 2H).

### Example 38

### N-(1H-Pyrrol-2-ylmethyl)-N′-(2-quinolinyl)-1,3-propanediamine

This compound was prepared from N-quinolin-2-yl-1,3-propanediamine and pyrrole-2-carboxaldehyde, and purified using HPLC (95% 0.1 M ammnium acetate buffer:5% CH₃CN→100% CH₃CN, 10 min 25 ml/min.) to give the title compound in 61% yield. ¹H NMR (400 MHz, MeOH-d₄) δ 7.86 (d, J=9.3 Hz, 1H), 7.61 (dd, J=1.7, 9.8 Hz, 1H), 7.46-7.42 (m, 1H), 7.22-7.18 (m, 2H), 6.81 (dd, J=1.5, 4.3 Hz, 1H), 6.75 (d, J=8.9 Hz, 1H), 6.22 (dd, J=1.8, 5.0 Hz, 1H), 6.13 (t, J=3.2 Hz, 1H), 4.18 (s, 2H), 3.66 (t, J=6.3 Hz, 2H), 3.03 (t, J=6.8 Hz, 2H), 2.04 (quintet, J=6.5 Hz, 2H).

### Example 39

### N-[3-(5-Methyl-2-furanyl)butyl]-N′-(2-quinolinyl)-1,3-propanediamine

This compound was prepared from N-quinolin-2-yl-1,3-propanediamine and 3-(5-methyl-2-furyl)-butyraldehyde, and purified using HPLC (95% 0.1 M nium acetate buffer.5% CH₃CN→100% CH₃CN, 10 min 25 ml/min.) to give the title compound in 19% yield. ¹H NMR (400 MHz, MeOH-d₄) δ 7.86 (d, J=8.9 Hz, 1H), 7.62 (dd, J=1.2, 9.3 Hz, 2H), 7.58 (d, J=8.5 Hz, 2H), 7.53-7.49 (m, 2H), 7.24-7.20 (m, 1H), 6.76 (d, J=9.1 Hz, 1H), 5.86 (d, J=3.0 Hz, 2H), 5.84-5.83 (m, 2H), 3.62 (t, J=6.4 Hz, 2H), 2.98 (t, J=6.7 Hz, 2H),2.92-2.75 (m, 4H), 2.18 (d, J=0.8 Hz, 3H), 1.98 (quintet, J=6.6 Hz, 3H), 1.90 (s, 3H), 1.87-1.78 (m, 4H).

### Example 40

### N-[(5-Nitro-3-thienyl)methyl]-N′-(2-quinolinyl)-1,3-propanediamine

This compound was prepared from N-quinolin-2-yl-1,3-propanediamine and 5-nitrothiophene,3-carboxaldehyde, and purified using HPLC (95% 0.1 M amnonium acetate buffer:5% CH₃CN→100% CH₃CN, 15 min 25 ml/min.) to give the title compound in 64% yield. ¹H NMR (400 MHz, MeOH-d₄) δ 7.94 (d, J=1.7 Hz, 1H), 7.87 (d, J=9.1 Hz, 1H), 7.78 (d, J=1.0 Hz, 1H), 7.61 (d, J=8.5 Hz, 1H), 7.48-7.40 (m, 2H), 7.20 (t, J=7.4 Hz, 1H), 6.76 (d, J=8.8 Hz, 1H), 4.11 (s, 2H), 3.64 (t, J=6.6 Hz, 2H), 3.03 (t, J=6.8 Hz, 2H), 2.04 (quintet, J=6.6 Hz, 2H).

### Example 41

### N-(6-Methoxy-4-methyl-2-quinolinyl)-N′-[(5-nitro-3-thienyl)methyl]-1,3-propanediamine

This compound was prepared from N-(6-methoxy-4-methyl-2-quinolinyl)-1,3-propanediamine and 5-nitrothiophene-3carboxaldehyde, and purified using HPLC (95% 0.1 M ammonium acetate buffer:5% CH₃CN→100% CH₃CN, 10 min 25 ml/min.) to give the title compound in 63% yield ¹H NMR (400 DMF-d₇) δ 8.09 (d, J=1.8 Hz, 1H), 7.87-7.87 (m, 1H), 7.46 (d, J=8.9 Hz, 1H), 7.19 (d, J=2.8 Hz, 1H), 7.15 (dd, J=2.8, 11.7 Hz, 1H), 6.67 (d, J=1.0 Hz, 1H), 3.88 (s, 3H), 3.78 (s, 2H), 3.54 (t, J=6.6 Hz, 2H), 2.70 (t, J=6.7 Hz, 2H), 2.49 (d, J=1.0 Hz, 3H), 1.82 (quintet, J=6.7 Hz, 2H).

### Example 42

### N-(6-Methoxy4methyl-2-quinolinyl)-N′-(1H-pyrrol-2-ylmethyl)-1,3-propanediamine

This compound was prepared from N-(6methoxy-4-methyl-2quinolinyl)-1,3-propanediamine and pyrrole-2-carboxaldehyde, and purified using HPLC (95% 0.1 M ammonium acetate buffer:5% CH₃CN→100% CH₃CN, 10 min 25 ml/mim) to give the title comupound in 83% yield. ¹H NMR (400 MHz, DMP-d₇) δ 7.56 (d, J=8.9 Hz, 1H), 7.36 (d, J=2.8 Hz, 1H), 7.31 (dd, J=3.0, 11.9 Hz, 1H), 6.95-6.93 (m, 1H), 6.86 (d, J=0.8 Hz, 1H), 6.19-6.17 (m, 1H), 6.15-6.13 (m, 1H), 4.13 (s, 2H), 4.05 (s, 3H), 3.71 (t, J=6.5 Hz, 2H), 2.99 (t, J=6.9 Hz, 2H), 2.66 (d, J=0.8 Hz, 3H), 2.11-2.10 (m, 2H).

### Example 43

### N-[(3,4-Dichlorophenyl)methyl]-N′-methyl-N′-2quinolinyl)-1,3-propanediamine

The title compound was isolated from t synthesis of Example 22. ¹H NMR (400 MHz, MeOH-d₄) δ 7.93 (d, J=9.3 Hz, 1H)), 7.60 (d, J=7.7 Hz, 1H), 7.45-7.37 (m, 3H), 7.32 (d, J=8.3 Hz, 1H), 7.18-7.14 (m, 1H), 7.09 (dd, J=2.0, 10.3 Hz, 1H), 6.99 (d, J=9.3 Hz, 1H), 3.83 (t, J=6.7 Hz, 2H), 3.65 (s, 211), 3.12 (s, 3H), 2.58 (t, J=6.7 Hz, 2H), 1.91 (quintet, J=7.0 Hz, 2H).

### Example 44

### N-[1-(2,5-Diethyl-3-thienyl)ethyl]-N′-(2-quinolinyl)-1,3-propanediamine

This compound was prepared from N-quinolin-2-yl-1,3-propanediamine and 3-acetyl-2,5-dimethylthiophene, but subjected to microwave beating single node 120° C., 10 min., and purified on SiO₂ (CH₂Cl₂:MeOH 10:0→4:1) to give the title compound in 26% yield. ¹H NMR (400 MHz, MeOH-d₄) δ 7.84 (d, J=9.1 Hz, 1H), 7.61 (dd, J=1.7, 9.7 Hz, 1H), 7.49-7.45 (m, 1H), 7.33 (d, J=9.1 Hz, 1H), 7.21 (t, J=7.8 Hz, 1H), 6.72 (d, J=9.4 Hz, 1H), 6.43 (s, 1H), 4.40 (q, J=6.9 Hz, 1H), 3.71-3.55 (m, 2H), 2.99-2.83 (m, 2H), 2.27 (s, 3H), 2.25 (s, 3H), 2.06-1.95 (m, 2H), 1.91 (s, 3H).

### Example 45

### N-[1-(2,5-Dichloro-thiophen-3-yl)-ethyl]-N′-(2-quinolinyl)-1,3-propanediamine

This compound was prepared from N-quinolin-2-yl-1,3-propanediamine and 1-(2,5-dichloro-thiophen-3-yl)-ethanone, but subjected to microwave heating single node 120° C., 5 min., and purified on SiO₂ (CH₂Cl₂:MeOH 10:0→4:1) to give the title compound in 11% yield. ¹H NMR (400 MHz, MeOH-d₄) δ 7.79 (d, J=8.8 Hz, 1H), 7.56 (bt, J=8.0 Hz, 2H), 7.49-7.44 (m, 1H), 7.16 (dt, J=1.2, 7.4 Hz, 1H), 6.70 (s, 1H), 6.69 (bd, J=9.0 Hz, 1H), 3.93 (q, J=6.7 Hz, 1H), 3.59 (m, 1H), 3.47 (m, 1H), 2.50 (m, 2H), 1.81 (m, 2H), 1.28 (d, J=6.9 Hz, 3H).

### Example 46

### N-[(1-acetyl-1H-indol-3-yl)methyl]-N′-Quinolin-2-ylcyclohexane-1,3-diamine

A solution of N-quinolin-2-ylcyclohexane-1,3-diamine (1.01 mmol, 0.243 g) and 1-acetyl-1H-indole-3-carboxaldehyde (0.63 mmol, 0.118 g) in MeOH:CH₂Cl₂ (1:2, containing 1% HOAc, 9 mL) was stirred at ambient temperature for 1 h, after which a solution of NaBH₃CN (2.50 mmol, 0.16 g) in MeOH (1.5 mL) was added. The reaction mixture was stirred at room temperature until LC/MS indicated that starting material was consumed. Methanol (10 mL) was added and the reaction mixture was concentrated. The residue was purified on SiO eluted with CH₂Cl₂:MeOH (95:5) and finally CH₂Cl₂:MeOH (9:1) to give 0.095 g (37%) of the title compound as a diastereomeric mixture (approx 3:1). ¹H NMR (400 MHz, MeOH-d₄) δ 8.36 (d, J=8.1 Hz, 1H, major isomer), 8.32 (d, J=8.3 Hz, 1H, minor isomer), 7.77 (d, J=8.9 Hz, 1H), 7.63-7.12 (m, 8H), 6.73 (d, J=8.9 Hz, 1H, minor isomer), 6.69 (d, J=8.9 Hz, 1H, major isomer), 4.42 (m, 1H, minor isomer), 4.06-3.96 (m, 1H, major isomer), 3.97 (s, 2H, major isomer), 3.96 (s, 2H, minor isomer), 3.00 (m, 1H, major isomer), 2.82 (tt, J=11.2, 3.6 Hz, 1H, major isomer), 2.60 (s, 3H, major isomer), 2.50-2.42 (m, 1H), 2.46 (s, 3H, minor isomer), 2.14-1.09 (m, 7H). ¹³C NMR (75 MHz, DMSO-d₆) δ (mixture of isomers) 168.4, 156.0, 148.0, 137.4, 137.2, 136.0, 129.8, 129.4, 127.3, 125.9, 125.3, 123.5, 123.2, 122.7, 121.8, 121.5, 119.0, 118.8, 116.7, 111.6, 111.0, 55.4, 52.3,48.5, 46.0, 42.0, 41.8, 39.5, 32.7, 32.6, 31.7, 23.9, 22.1, 19.9.

LC-MS [M+H]⁺ 413

### Example 47

### (1S,3S)-N-(6-methoxy-4methylquinolin-2-yl)-N′-(3-thienylmethyl)cyclopentane-1,3-diamine

### a) tert-butyl {(1S,3S)-3-[(6-methoxy-4-methylquinolin-2-yl)amino]cyclopentyl}carbamate

A mixture of 2-chloro-6-methoxy-4methylquinolin (3.33 mmol, 0.690 g), tert-butyl [(1S,3S)-3-aminocyclopentyl]carbamate (5.0 mmol, 1.00 g), NaO^{t}Bu (4.66 mmol, 0.45 g), Pd(OAc)₂ (0.33 mmol, 0.075 g), and BINAP (0.33 mmol, 0.207 g) in toluene (30 mL) was stirred at 100° C. under nitrogen until LC/MS indicated that staring material was consumed. The reaction mixture was cooled to room temperature, poured into EtO₂ (300 mL) and washed with brine. The organic layer was then separated, dried over Na₂SO₄ and evaporated to dryness. The residue was purified on a SiO2 column eluted with CH₂Cl₂:MeOH (95:5) to give 0.618 g (50%) of the tide compound.

LC-MS [M+2H]⁺ 373

### b) (1S,3S)-3-[(6-methoxy-4-methylquinolin-2-yl)cyclopentane-1,3-diamine

Tert-butyl {(1S,3S)-3-[(6-methoxy-4-methylquinolin-2-yl)-amino}cyclopentyl)carbamate (1.48 mmol 0.550 g) and TFA (3 mL) in CHCl₃ (7 mL) was stir at rt. for 6 hours. LC indicated that staring material was consumed. The mixture was then evaporated to dryness. pH was set to 10 with a 2 N NaOH solution and then extracted with EtOAc. The organic layer was separated, dried on MgSO₄ and concentrated, to give 0.400 g (99%) of the title compound ¹H NMR (300 MHz, CDCl₃) δ 7.57 (d, 1H), 7.16-7.20 ( dd 1H), 7.04 (d, 1H), 6.51 (s, 1H), 5.24 (br, 1H), 4.44 (m, 1H), 3.86 (s, 3H), 3.50 (m, 1H), 2.73 (br, 2H), 2.51 (s, 3H), 2.26 (m, 2H), 2.06 (m, 1H), 1.85 (m, 1H), 1.41 (m, 2H).

### c) (1S,3S)-N-(6-methoxylmethylquinolin-2-yl)-N′-(3-thienylmethyl)cyclopentane-1,3-diamine

(1S,3S)-N-(6-methoxy-4-methylquinolin-2-yl)cyclopentane-1,3-diamine (0.74 mmol 0.200 g) and thiophene-3-carboxylaldehyde (0.74mmol, 0.083 g) in MeOH:CH₂Cl₂ (1:1, containing 1% HOAc, 5 mL) was stirred at ambient temperature for 1 h, after which a solution of NaBH₃CN (1.48 mmol, 0.093 g) in MeOH (1 mL) was added. The reaction mixture was stirred at room temperature until LC-MS indicated that starting material was consumed. Methanol (5 mL) was added and the reaction mixture was concentrated. The residue was dissolved in MeCN and filtrated. The filtrate was then evaporated to dryness, dissolved in MeCN (10 mL) and purified by prep. HPLC (H₂O:MeCN) to give 0.180 g (95%) of the tide compound. ¹H NMR (300 MHz, CDCl₃) δ 7.58 (d, 1H), 7.27-7.29 (m, 1H), 7.19-7.23 (dd, 1H), 7.13 (d, 1H), 7.04-7.08 (m, 2H), 6.53 (s, 1H), 4.75 (br, 1 H), 4.38 (m, 1H), 3.89 (s, 3H), 3.80 (s, 2H), 3.33-3.38 (m, 1H), 2.54 (s, 3H), 2.31 (m, 1H), 1.95-2.08 (m, 2H), 1.85 (m, 1H), 1.49-1.53 (m, 2H). ¹³C NMR (CDCl₃) δ 155.6, 155.1, 144.9, 32.9, 32.2, 19.6.

MS (ESI) 368 (M +H⁺).

### Example 48

### (1s,3S)-N-(6-methoxy-4methylquinolin-2-yl)-N′-[(1-methyl-1H-indol-3 yl)methyl]cyclopentane-1,3-diamine

(1S,3S)-N-(6-methoxy-4-methylquinolin-2-yl)cyclopentane-1,3-diamine (0.74 mmol, 0.200 g) and 1-Methyl indol-3-carboxyaldehyde (0.74mmol, 0.118 g) in MeOH:CHCl₂ (1:1, containing 1% HOAc, 5 mL)was sted at ambient temperature for 1 h, after which a solution of NaBH₃CN (1.48 mmol, 0.093 g) in MeOH (1 mL) was added. The reaction mixture was stirred at room temperature until LC-MS indicated that starting material was consumed. Methanol (5 mL) was added and the reaction mixture was concentrated. The residue was dissolved in MeCN and filtrated. The filtrate was then evaporated to dryness, dissolved in MeCN (10 mL) and purified by prep. HPLC (H₂O:MeCN) to give 0.050 g (16%) of the title compouud. ¹H NMR (300 MHz, CDCl₃) δ 7.61-7.68 (m, 2H), 7.25-7.30 (m, 3H), 7.10-7.15 (m, 2H), 7.03 (s, 1H), 6.56 (s, 1H), 4.90 (br, 1H), 4.40-4.44 (q, 1 H), 3.98 (s, 2H), 3.81 (s, 3H), 3;48 (s, 3H), 3.44-3.48 (m, 1H), 2.56 (s, 3H), 2.31-2.35 (m, 1H), 2.02-2.10 (m, 2H), 1.84-1.91 (m, 1H), 1.54-1.60 (n, 2H). ¹³C NMR (CDCl3) δ 155.4, 154.8, 144.6, 143.0, 137.2, 127.6, 127.5, 123.9, 121.8, 120.4, 119.1, 118.9, 113.2, 111.0, 109.4, 103.7, 57.4, 55.7, 52.1,43.2, 40.8, 32.7, 32.6, 31.8, 19.3.

LC-MS [M+H]⁺ 415.

### Example 49

### N-[(1-acetyl-1H-indol-3-yl)methyl]-N′-(6-methoxy-4-methylquinolin-2-yl)cyclohexane-1,3-diamine

To a stirred solution of N-(6-methoxy-4-methylquinolin-2-yl)cyclohexane-1,3-diamine (0.526 mmol, 0.150 g) and 1-acetyl-1H-indole-3-carboxaldehyde (0.53 mmol, 0.098 g) in CH₂Cl₂MeOH 2:1 containing 1%-HOAc (5 mL), sodium cyanoborohydride (0.89 mmol, 0.056 g) was added. After 24 h, the mixture was concentrated and purified by flash chromatography, to give 0.119 g (50%) of the major diastereomer of the title conpoumd. ¹H NMR (400 MHz, CDCl₃) δ 8.43 (d, J=8.1 Hz, 1H), 7.61-7.57 (m, 2H), 7.37-7.26 (m, 3H), 7.19 (dd, J=9.1, 2.8 Hz, 1H), 7.06 (d, J=12.8 Hz, 1H), 6.42 (s, 1H), 4.88 (br, 1H), 4.04-3.95 (m, 3H), 3.86 (m, 3H), 2.82 (m, 1H), 2.58 (s, 3H), 2.48 (s, 3H), 2.44-2.38 (m, 1H), 2.11-1.82 (m, 4H), 1.52-1.11 (m, 4H); ¹³C NMR (101 MHz, CDCl₃) δ 168.6, 155.0, 154.7, 144.0, 143.5, 136.2, 130.0, 127.9, 125.4, 124.1, 123.7, 122.7, 121.9, 120.0, 119.0, 116.8, 112.1, 103.8, 55.7 (2C), 48.7, 42.1, 40.1, 33.1, 32.8, 24.1, 22.4, 19.i; LC-MS [M+H]⁺ 457.3.

A minor diastereomer was isolated and firer purified by HPLC (95% 0. 1M anmonium acetate buffer:5% CH₃CN→100% CH₃CN, 10 ml/min) to give 0.027 g (11%) of the minor diastereomer of the title compound. ¹H NMR (500 Mz, CDCl₃) δ 8.43 (bs, 1H), 7.62 (d, J=7.5 Hz, 1H), 7.57 (d, J=9.1 Hz, 1H), 7.37-7.25 (m, 3H), 7.18 (d, J=8.3 Hz, 1H), 7.07 (s, 1H), 6.50 (s, 1H), 4.69 (s, 1H), 4.29(s, 1), 4.01 (d, J=13.6 Hz, 1H), 3.96 (d, J=13.6 Hz, 1H), 3.88 (s, 3H), 3.03 (bs, 1H), 2.53 (s, 3H), 2.52 (s, 3H), 1.92-1.4 (m, 9H); LC-MS [M+H]⁺ 457.3.

### Example 50

### N-(1H-indol-3-ylmethyl)-N′-(6-methoxy-4-methylquinolin-2-yl)cyclohexane-1,3-diamine

The title compound was isolated from synthesis of Example 49 and further purified by HPLC (95% 0.1M ammonium acetate buffer.5% CH₃CN→100% CH₃CN, 10 ml/min) to give 0.013 g (6%) of the title compound as a single diastereomer. ¹H NMR (400 MHz, MeOH-d₄) δ 7.62 (d, J=7.9 Hz, 1H), 7.53 (d, J=9.1 Hz, 1H), 7.36 (d, J=8.3 Hz, 1H), 7.26 (s, 1H), 7.17-7.09 (m, 3H), 7.06 (m, 1H), 6.57 (s, 1H), 4.05 (s, 2H), 3.92 (tt, J=11.4, 3.8 Hz, 1H), 3.86 (s, 3H), 2.86 (tt, J=11.3, 3.7 Hz, 1H), 2.49 (s, 3H), 2.47-2.41 (m, 1H), 2.08-2.02 (m, 1H), 1.90-1.82 (m, 1H), 1.52-1.40 (m, 1H), 1.24-1.11 (m, 3H); LC-MS [M+H]⁺ 415.3.

### Example 51

### N-(6-methoxy-4-methylquinolin-2-yl)-N′-(3-thienylmethyl)cyclohexane-1,3-diamine

N-(6-methoxy-4-methylquinolin-2-yl)cyclohexane-1,3 e (0.16 mmol, 0.046 g) in CH₂Cl₂/MeOH 1:1 (1.2 mL), thiophene-3carboxaldehyde (0.12 mmol, 0.014 g) in CH₂Cl₂ (0.6 mL) and HOAc (0.060 mL) was added to Pol-BH₃CN (150 mg, pre-swollen in CH₂Cl₂, 0.6 mL). The resultant slurry was subjected to microwave heating single node 100° C., 5 min. The resin was filtered and washed with portions (1-2 mL) of CH₂Cl₂ and MeOH, and the filtrate was concentrated. The residue was purified on HPLC (95% 0.1M ammonium acetate buffer:5% CH₃CN→100% CH₃CN, 10 ml/min) to give 0.021 g (39%) of the title compound as a mixture of diastereomers (˜5:1). ¹H NMR (400 MHz, MeOH-d₄) δ 7.56 (m, 1H, rninor isomer), 7.55 (d, J=9.1 Hz, 1H, major isomer), 7.44-7.40 (m, 211), 7.33 (dd, J=5.0, 3.0 Hz, 1H, minor isomer), 7.25 (m, 1H, minor isomer), 7.19-7.13 (m, 3H) 7.07 (dd, J=5.0, 1.2 Hz, 1H, minor isomer), 6.66 (bs, 1H, minor isomer), 6.59 (bs, 1H, major isomer), 4.36 (m, 1H, minor isomer), 4.02 (s, 2H, major isomer), 4.01 (s, 2H, minor isomer), 3.94 (tt, J=11.5, 3.7 Hz, 1H, major isomer), 3.87 (s, 3H, major isomer), 3.86 (s, 3H, major isomer), 3.10 (m, 1H, minor isomer), 2.94 (tt, J=11.6, 3.7 Hz, 1H, major isomer), 2.52-2.46 (m, 1H, major isomer), 2.52 (s, 3H, minor isomer), 2.50 (s, 3H, major isomer), 2.34-2.28 (m, 1H, minor isomer), 2.12-1.15 (m, 7H); ¹³C NMR (101 MHz, MeOH-₄, major isomer) δ 156.6, 156.2, 145.7, 143.7, 137.9, 129.0, 127.5, 127.3, 125.4, 125.2, 120.9, 114.3, 104.9, 56.3, 56.0, 49.3, 45.1, 38.9, 33.6, 31.4, 23.9, 18.9; LC-MS [M+H⁺ 382.2.

### Example 52

### N-(6-methoxy-4-methylquinolin-2-yl)-N′-[(1-methyl-1H-indol-3-yl)methyl]cyclohexane-1,3diamine

N-(6-methoxymethylquinolin-2-yl)cyclohexane-1,3-diamine (0.16 mmol, 0.046 g) in CH₂Cl₂/MeOH 1:1(1.2 mL), 1-methylindole-3-carboxaldehyde (0.13 mmol, 0.021 g) in CH₂Cl₂ (0.6 mL) and HOAc (0.060 mL) was added to Pol-BH₃CN (150 mg, pre-swollen in CH₂Cl₂, 0.6 mL). The resultant slurry was subjected to microwave beating single node 100° C., 10 min. The resin was filtered and washed with portions (1-2 mL) of CH₂Cl₂ and MeOH and the filtrate was concentrated. The residue was purified on HPLC (95% 0.1M ammonium acetate buffer:5% CH₃CN→100% CH₃CN, 10 mL/min) to give 0.021 g (34%) of the title comzpound as a mixture of diastereomers (˜6:1). ¹H NMR (400 MHz, MeOH-d₄) δ 7.65 (d, J=8.1 Hz, 1H, major isomer), 7.59-7.55 (m, 1H, minor isomer), 7.54 (d, J=9.1 Hz, 1H, major isomer), 7.37 (d, J=8.3 Hz, 1H, major isomer), 7.30 (d, J=8.3 Hz, 1H, minor isomer), 7.27 (s, 1H, major isomer), 7.23-7.07 (m, 5H), 7.01-6.97 (m, 1H, minor isomer), 6.62 (s, 1H, minor isomer), 6.58 (s, 1H, major isomer), 4.36 (m, 1H, minor isomer), 4.20 (s, 2H), 3.95 (tt, J=11.4, 3.7 Hz, 1H, major isomer), 3.87 (s, 3H, minor isomer), 3.85 (s, 3H, major isomer), 3.78 (s, 3H, major isomer), 3.59 (s, 3H, minor isomer), 3.21 (m, 1H, minor isomer), 3.07 (tt, J=11.5, 3.4 Hz., 1H, major isomer), 2.58-2.40 (m, 1H), 2.51 (s, 3H, major isomer), 2.49 (s, 3H, major isomer), 2.18-1.19 (m, 7H); LC-MS [M+H⁺ 429.3.

### Example 53

### N-(1-benzofuran-2-ylmethyl)-N′-(6-methoxy-4-methylquinolin-2-yl)cyclohexane-1,3-diamine

N-(6-methoxy-4-methylquinolin-2-yl)cyclohexane-1,3-diamine (0.14 mmol, 0.040 g) in CH₂Cl₂/MeOH 1:1 (1.2 mL), benzofuran-2-carboxaldehyde (0.13 mmol, 0.018 g) in CH₂Cl₂ (0.6 mL) and HOAc (0.060 mL) was added to Pol-BH₃CN (150 mg, pre-swollen in CH₂Cl₂, 0.6 mL). The resultant slurry was subjected to microwave heating single node 100° C., 10 min. The resin was filtered and washed with portions (1-2 mL) of CH₂Cl₂ and MeOH, and the filtrate was concentrated. The residue was purified on a Biotage Horizon 25 mm silica columm (linear gradient EtOAc/MeOH 19:1, containing 1% NEt₃→EtOAc/MeOH 1:1, containing 1% NEt₃, 10 ml/min) to give 0.015 g (26%) of the title comnpound as a mixture of diastereomers (˜10:1). ¹H NMR (400 MHz, MeOH-d₄) δ 7.54-7.10 (m, 7H), 6.68 (s, 1H, major isomer), 6.61 (s, 1H, minor isomer), 6.57 (s, 1H, major isomer), 6.47 (s, 1H, minor isomer), 4.31 (m, 1H, minor isomer), 3.95 (s, 2H), 3.95-3.85 (m 1H, major isomer), 3.85 (s, 3H), 2.89 (m, 1H, minor isomer), 2.72 (tt, J=11.2, 3.6 Hz, 1H, major isomer), 2.48 (s, 3H), 2.40-2.34 (m, 1H), 2.06-1.05 (m, 7H); LC-MS [M+H⁺ 416.2.

### Example 54

### N-(6-methoxy-4-methylquinolin-2-yl)-N′-(pyridin-2ylmethyl)cyclohexane-1,3-diamine

N-(6-methoxy-4-methylquinolin-2-yl)cyclohexane-1,3-diamine (0.14 mmol, 0.040 g) in CH₂Cl₂/MeOH 1:1 (1.2 mL), pyridin-2-carboxaldehyde (0.13 mmol, 0.014 g) in CH₂Cl₂ (0.6 mL) and HOAc (0.060 mL) was added to Pol-BH₃CN (150 mg, pre-swollen in CH₂Cl₂, 0.6 mL). The resultant slurry was subjected to microwave heating single node 100° C., 10 min. The resin was filtered and washed with portions (1-2 mL) of CH₂Cl₂ and MeOH, and the filtrate was concentrated The residue was purified on a Biotage Horizon 25 mm silica column (linear gradient EtOAc/MeOH 19:1, containing 1% NEt₃→EtOAc/MeOH 1:1, containing 1% NEt₃, 10 ml/min) to give 0.015 g (45%) of the title compound as a mixture of diastereomers (˜10:1). ¹H NMR (400 MHz, MeOH-hd4) δ 8.49 (m, 1H, major isomer), 8.42 (m, 1H, minor isomer), 7.78 (td, J=7.7, 1.8 Hz, 1H, major isomer), 7.65 (td, J=7.7, 1.8 Hz, 1H, minor isomer), 7.52 (d, J=9.1 Hz, 1H, major isomer), 7.44 (d, J=7.9 Hz, 1H, major isomer), 7.37 (d, J=7.9 Hz, 1H, minor isomer), 7.30-7.27 (m, 1H), 7.23-7.08 (m, 2H), 6.64 (bs, 1H, minor isomer), 6.57 (bs, 1H, major isomer), 4.36 (m, 1H, minor isomer), 3.95-3.87 (m, 1H, major isomer), 3.92 (s, 2H), 3.86 (s, 3H, minor isomer), 3.85 (s, 3H, major isomer), 3.29 (m, 1H, minor isomer), 2.69 (tt, J=11.2, 3.7 Hz, 1H, major isomer), 2.50 (s, 3H, minor isomer), 2.49 (s, 3H, major isomer), 2.40-2.32 (m, 1H), 2.08-1.98 (m, 2H), 1.88-1.07 (m, 5H); LC-MS [M+H]⁺ 3.77.2.

### Example 55

### N-(4-methylquinolin-2-yl)-N′-(3-thienymethyl)cyclohexne1,3-diamine

N-(4methylquinolin-2-yl)cyclohexane-1,3-diamine (75 mg, 0.29 mmol) in 2 mL of CH₂Cl₂/MeOH 1:1, and 3-thiophenaldehyde (26 mg, 0.23 mmol) in 1 mL of CH₂Cl₂, and 0.10 mL of acetic acid were added to Pol-BH₃CN (0.25 g, preswollen in 1 mL of CH₂Cl₂). The resultant slurry was subjected to single node microwave beating (100° C. for 10 min). The resin was filtered and washed with 1-2 mL portions of CH₂Cl₂ and MeOH. The filtrates were combined and poured onto a 1 g SCX-2 prepacked ion-exchange column, washed with 10 mL of MeOH and the product was eluted with MeOH containing 10% of Et₃N. The purity was not satisfactory and the product was further purified on a Biotage Horizon 12 mm silica column (linear gradient EtOAc/MeOH 9:1→EtOAc/MeOH 1:1, 10 mL/min) to give 20 mg (19%) of the title compound as a mnixture of diastereomers (˜3:1). ¹H NMR (300 MHz, MeOH-₄) δ 7.68-7.75 (m, 1H), 7.5-7.6 (m, 1H), 7.0-7.5 (m, 5H), 6.61 (bs, 1H, minor isomer), 6.54 (bs, 1H, major isomer), 4.36 (m, 1H, minor-isomer), 4.11 (s, 2H, major isomer), 4.09 (s, 2H, minor isomer), 3.95 (m, 1H, major isomer), 3.09 (m, 1H, major isomer; minor isomer obscured under the MeOH-d₄ signal), 2.35-2.6 (m, 4H; thereof 2.48, 3H, minor isomer, and 2.46, 3H, major isomer), 1.1-2.2 (m, 7H).

¹³C NMR (75 MHz, MeOH-₄, major isomer) δ 179.4, 157.3, 148.0, 146.7, 134.7, 130.4, 128.9, 128.0, 127.0, 125.6, 124.8, 123.0, 113.9, 68.1, 56.4, 44.2, 37.5, 33.1,30.2, 23.8, 18.8.

LC-MS M+]⁺ 352.3.

### Appendix

Names/Reference Numbers of Staring Materials

Commercial starting material (CAS no): 2-chloroquinoline, 612-62-4; 2-chloro-6 methoxy-4-methylquinoline, 6340-55-2; 1,3-diaminopropane, 109-762; ethylenediamine, 107-15-3; 1,3-cyclohexanediamine, 3385-21-5; 1,4-cyclohexanediamine, 3114-70-3; 4-aminopiperidine, 13035-19-3; N-methyl-1,3-propanediamine, 6291-84-5; 3-thiophenecarboxaldehyde, 498-62-4; 3-acetylthiophene, 1468-83-3; 4-keto-4,5,6,7-tetrahydrothianaphthene, 13414-95-4; 3-acetylthianaphthene, 1128-05-8; 2-thiophenecarboxaldehyde, 98-03-3; 5-nitrothiophene-3-carboxaldehyde, 75428-45-4; 3-acetyl-2,5-dimethylthiophene, 2530-10-01; 1-acetyl-3-indolecarboxaldehyde, 22948-94-3; indole-3-carboxaldehyde, 487-89-8; pyrrole-2-carboxaldehyde, 1003-29-8; 2,4,6-methyl-benzaldehyde, 487-68-3; phenylacetaldehyde, 122-78-1; 3,4-dichlorobenzaldehyde, 6287-38-3; 2-naphthaldehyde, 66-99-9; 2-quinolinecarboxaldehyde, 5470-96-2; diphenylacetaldehyde, 947-91-1; 4-biphenylcarboxaldehyde, 3218-36-8; 4-dimethylaminobenzaldehyde, 100-10-7; 3-furaldehyde, 498-60-2; 3-(5-methyl-2-furyl)butyraldehyde, 31704-80-0; cyclopropanecarboxaldehyde, 1489-69-6; 1-methylindole-3-carboxaldehyde, 19012-03-4; benzofuran-2-carboxaldehyde, 4265-16-1; pyridin-2-carboxaldehyde, 1121-60-4 3-acetyl-2,5-dichlorothiophene, 36157-40-1; (−)-2-azabicyclo[2.2.1]hept-5-en-3-one,79200-56-9 and 2-chloro-4-methylquinoline 634-47-9

Pharmacological Properties

MCH1 Receptor Radioligand Binding.

Assays were performed on membranes prepared from HEK293 cells stably expressing the human Melanin concentrating hormone receptor 1 (MCH1r) (Lembo et al. Nature Cell Biol 1 267-271). Assays were performed in a 96-well plate format in a final reaction volume of 200 μl per well. Each well contained 6,1 μg of membrane proteins diluted in binding buffer (50 mM Tris, 3 mM MgCl₂, 0.05% bovine serum albumin (BSA) and the radioligand ¹²⁵I-MCH (IM344 Amersham) was added to give 10 000 cpm (counts per Minute) per well Each well contained 2 μl of the appropriate concentration of competitive antagonist prepared in DMSO and left to stand at room temperature for 60 minutes. Non-specific binding was determined as that remaining following incubation with 1M MCH (Melanin concentrating hormone, H-1482 Bachem). The reaction was terminated by transfer of the reaction to GF/A filters using a Micro96 Harvester (Skatron Instruments, Norway). Filters were washed with assay buffer; Radioligand retained on the filters was quantified using a1450 Microbeta TRILUX (Wallac, Finland).

Non-specific binding was subtracted from all values determined. Maximum binding was that determined in the absence of any competitor following subtraction of the value determined for non-specific binding. Binding of compounds at various concentrations was plotted according to the equation
y=A+(B−A)/1+((C/x)ˆD)))
and IC₅₀ estimated where

A is the bottom plateau of the curve i.e. the final minimum y value

B is the top of the plateau of the curve i.e. the final maximum y value

C is the x value at the middle of the curve. This represents the log EC50 value when A+B=100

D is the slope factor.

x is the original known x values.

y is the original known y values.

The compounds exemplified herein had an IC₅₀ of less than 2 μmolar in the above assay. Preferred compounds had an activity of less than 1 μmolar. For Example the IC₅₀s of Examples 2, 29 and 53 were 0.01, 0.40 and 0.56 μmol, respectively.

Assays were also performed on membranes prepared from HEK293 cells stably expressing the rat Melanin concentrating hormone receptor 1 (MCH1r) (Lembo et al. Nature Cell Biol 1 267-271). Assays were performed in a 96-well plate format in a final reaction volume of 200 μl per well. Each well contained 5 μg of membrane proteins diluted in binding buffer (50 m/M Tris, 3 mM MgCl₂, 0.05% bovine seruma albumin (BSA) and the radioligand ¹²⁵I-MCH (IM344 Amersham) was added to give 10 000 cpm (counts per minute) per well. Each well contained 2 μl of the appropriate concentration of competitive antagonist prepared in DMSO and left to stand at room temperature for 60 minutes. Non-specific binding was determined as that remaining following incubation with 1 μM MCH (Melanin concentrating hormone, H-1482 Bachem). The reaction was terminated by transfer of the reaction to GF/A filters using a Micro96 Harvester (Skatron Instruments, Norway). Filters were washed with assay buffer. Radioligand retained on the filters was quantified using a1450 Microbeta TRILUX (Wallac, Finland).

1. A compound of formula (I) 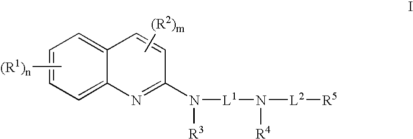
wherein
R¹ represents a C₁₋₄alkoxy group optionally substituted by one or more fluoro or a C₁₋₄alkyl group optionally substituted by one or more fluoro;
n represents 0 or 1;
R¹ represents a C₁₋₄alkyl group optionally substituted by one or more fluoro or a C₁₋₄alkoxy group optionally substituted by one or more fluoro;
m represents 0 or 1;
R³ represents H or a C₁₋₄alkyl group;
L¹ represents au alkylene chain (CH₂)ᵣ in which r represents 2 or 3 or L¹ represents a cyclohexyl group wherein the two nitrogens bearing R³ and R⁴, respectively, are linked to the cyclohexyl group either via the 1,3 or the 1,4 positions of the cyclohexyl group or L¹ represents a cyclopentyl group wherein the two nitrogens bearing R³ and R⁴, respectively, are linked to the cyclopentyl group via the 1,3 position of the cyclopentyl group and additionally when R⁵ represents 9,10-methanoanthracen-9(10H)-yl the group -L¹-N(R⁴)— together represents a piperidyl ring which is linked to L² through the piperidinyl nitrogen and to N—R³ via the 4 position of the piperidyl ring with the proviso that when R⁵ represents 9,10-methanoanthracen-9(10H)-yl then r is only 2;
R⁴ represents H or a C₁₋₄alkyl group optionally substituted by one or more of the following: an aryl group or a heteroaryl group;
L² represents a bond or an alkylene chain (CH₂), in which s represents 1, 2 or 3 wherein the alkylene chain is optionally substituted by one or more of the following: a C₁₋₄alkyl group, phenyl or heteroaryl;
R⁵ represents aryl, a heterocyclic group or a C₃₋₈cycloalkyl group which is optionally fused to a phenyl or to a heteroaryl group; as well as optical isomers and racemates thereof as well as pharmaceutically acceptable salts, thereof;
with a first proviso that when n is 0, and m is 1 and R¹ is methyl located at the 4-position of the quinoline ring, and R³ is H and R⁴ is H and L¹ is (CH₂)₂ or (CH₂)₃ or 1,4cyclohexyl, and L² is a bond then R⁵ is not 4methylquinolin-2-yl;
and with a second proviso that when n is 0, and m is 0 or 1 and R² is a C₁₋₃alkoxy group located at the 4-position of the quinoline ring, and R³ is H or a C₁₋₃alkyl group and R⁴ is H or a C₁₋₃alkyl group and L¹ is (CH₂)₃ and L² is methylene optionally substituted by one or more C₁₋₃alkyl groups or phenyl then R⁵ is not phenyl, thienyl or indolyl optionally substituted by one, two or three C₁₋₄alkyl groups or halo.

2. A compound as claimed in claim 1 in which R¹ represents a C₁₋₄alkoxy group.

3. A compound as claimed in claim 1 or claim 2 in which R² represents a C₁₋₄alkyl group.

4. A compound as claimed in any previous claim in which L¹ represents triethylene, 1,3-cyclohexyl or 1,4cyclohexyl or when R⁵ represents 9,10-methanoanthracen-9(10H)-yl L¹ additionally represents ethylene.

5. A compound as claimed in any previous claim in which L¹ represents trimethylene.

6. A compound as claimed in any previous claim in which L¹ represents 1,3-cyclohexyl.

7. A compound as claimed in any previous claim in which L¹ represents 1,4cyclohexyl.

8. A compound as claimed in any previous claim in which L¹ represents 1,3-cyclopentyl.

9. A compound as claimed in any previous claim in which R³ represents H.

10. A compound as claimed in any previous claim in which L² represents methylene.

11. A compound as claimed in any previous claim in which R⁴ represents H.

12. A compound as claimed in any previous claim in which R⁵ represents phenyl 2-naphthyl or 9,10-methanoanthracen-9(10H)-yl, each of which is optionally substituted by one or more of the following: methyl, chloro, dimethylamino or phenyl.

13. A compound as claimed in any previous claim in which R⁵ represents 4,5,6,7-tetrahydrothianaphth-4yl, benzo[b]thien-3-yl 2-thienyl, 3-thienyl, 2-furyl, 3-furyl, benzofuranyl pyridyl, 1H-pyrrol-2-yl, 1H-indol-3-yl, or 2-quinolinyl, each of which is optionally substituted by one or more of the following: nitro, methyl, acetyl or chloro.

14. A compound selected from:
N-(9,10-methanoanthracen-9(10H)-ylmethyl)-N′-(2-quinolinyl) -1, 2-ethanediamine;
N-(6-methoxy-4methyl-2-quinolinyl)-N′-(3-thienylmethyl)-1,3-propanediamine;
N-(9,10-methanoanthracen-9(10H)-ylmethyl)-N′-(2-quinolinyl)-1,3-propanediamine;
N-(2-quinolinyl)-N′-(3-thienylmethyl)-1,3-propanediamine;
N-(9,10-methanoanthracen-9(10H)-ylmethyl)-N′-(2-quinolinyl)-1,4-cyclohexanediamine;
N-[(1-acetyl-1H-indol-3-yl)methyl]-N′-(6-methoxy-4-methyl-2-quinolinyl)-1,3-propanediamine;
N-(9,10-methanoanthracen-9(10H)-ylmethyl)-N′-2-quinolinyl-1,3-cyclohexanediamine;
N-(2-quinolinyl)-N′-[1-(3-thienyl)ethyl]-1,3-propanediamine;
N-(2-quinolinyl)-N′-(3-thienylmethyl)-1,3-cyclohexanediamine;
N-(9,10-methanoanthracen-9(10H)-ylmethyl)-N′-(6-methoxy-4-methyl-2-quinolinyl)-1,3-propanediamine;
N-(2-quinolinyl)-N′-(4,5,6,7-tetrahydrothianaphth-4-yl)-1,3-propanediamine;
N-methyl-N′-2-quinolinyl)-N-(3-thienylmethyl-1,3-propanediamine;
N-(2-quinolinyl-N′, N′-bis(3-thienymethyl)-1,3-propanediamine;
N-(9,10-methanoanthracen-9(10H)-ylmethyl)-N-methyl-N′-2-quinolinyl)-1,3-propanediamine;
N-(2-quinolinyl)-N′-(2,4,6-trimethylphenyl)methyl]-1,3-propanediamine;
N-(2-phenylethyl)-N′-(2-quinolinyl)-1,3-propanediamine;
N-(1-benzo[b]thien-3-ylmethyl)-N′-(2-quinolinyl-1,3-propanediamine;
N-[(3,4-dichlorophenyl)methyl-N′-(2-quinolinyl)-1,3-cyclohexanediamine;
N-(9,10-methanoanthracen-9(10H)-ylmethyl-N′-methyl-N′-2-quinolinyl)-1,3-propanediamine;
N-(2-quinolinyl)-N′-(2-thienylmethyl)-1,3-propanediamine;
N-(3-furanylmethyl)-N′-(2-quinolinyl)-1,3-propanediamine;
N-[(3,4-dichlorophenyl)methyl]-N-ethyl-N′-(2-quinolinyl)-1,3-propanediamine;
N-[1-(9,10-methanoanthracen-9(10H)-ylmethyl)-4-piperidyl]-2-quinolinamine;
N-(1H-indol-3-ylmethyl)-N′-2-quinolinyl)-1,3-propanediamine;
N-(2-naphthalenymethyl)-N′-(2-quinolinyl)-1,3-propanediamine;
N-(2,2-diphenylmethyl)-N′-(2-quinolinyl)-1,3-propanediamine;
N-(H-indol-3-ylmethyl)-N′-(6-methoxy-4-methyl-2-quinolinyl-1,3-propanediamine;
N-[(3,4-dichlorophenyl)methyl-N′-(2-quinolinyl)-1,3-propanediamine;
N-[(3,4-dichlorophenyl)methyl]-N′-(2-quinolinyl)-1,4-cyclohexanediamine;
N, N′-di-(2-quinolinyl)-1,3-propanediamine;
N-(2-quinoliiiyl)-N′-(2-quinolinylmethyl)-1,3-propanediamine;
N-[(1-acetyl-1H-indol-3-yl)methyl-N′-(2-quinolinyl)-1,3-propanediamine;
N-(cyclopropylmethyl)-N′-(2-quinolinyl)-1,3-propanediamine;
N-(2-quinolinyl)-N′-(3-thienylmethyl)-1,4-cyclohexanediamine;
N-(1,1-biphenyl]-4ylmethyl)-N′-(2-quinolinyl)-1,3-propanediamine;
N-(6-methoxymethyl-4-quinolinyl)-N′-[3-(5-methyl-2-furanyl)butyl]-1,3-propanediamine;
N-[]4-(dimethylamino)phenyl]methyl]-N′-2-quinolinyl)-1,3-propanediamine;
N-(1H-pyrrol-2-ylmethyl)-N′-(2-quinolinyl)-1,3-propanediamine;
N-[3-(5-methyl-2-furanyl)butyl]-N′-(2-quinolinyl)-1,3-propanediamine;
N-[(5-nitro-3-thienyl)methyl]-N′-2-quinolinyl)-1,3-propanediamine;
N-(6-methoxy-4-methyl-2-quinolinyl)-N′-[(5-nitro-3-thienyl)methyl-1,3-propanediamine;
N-(6-methoxy-4methyl-2-quinolinyl)-N′-(1H-pyrrol-2-ylmethyl)-1,3-propanediamine;
N-[(3,4dichlorophenyl)methyl]-N′-methyl-N′-2-quinolinyl)-1,3-propanediamine;
N-[1-(2,5-dimethyl-3-thienyl)ethyl]-N′-(2-quinolinyl)-1,3-propanediamine;
N-[1-(2,5-Dichloro-thiophen-3-yl)-ethyl]-N′-(2-quinolinyl)-1,3-propanediamine;
N-[(1-acetyl-1H-indol-3-yl)methyl]-N′-quinolin-2-ylcyclohexane-1,3-diamine;
N-(6-methoxy-4-methylquinolin-2-yl)-N′-(3-thienylmethyl)cyclopentane-1,3-diamine;N-(6methoxy-4-methylquinolin-2-yl)-N′-[(1-methyl-1H-indol-3-ylmethyl]cyclopentane-1,3-diamine;
(1S,3S)-N-(6-methoxy-4-methylquinolin-2-yl)-N′-[(1-methyl-1H-indol-3-yl)methyl]cyclopentane-1,3-diamine
(1 S,3S)-N-(6-methoxy-4-methylquinolin-2-yl)-N′-(3-thienylmethyl)cyclopentane-1,3-diamine
N-[(1-acetyl-1H-indol-3-yl)methyl]-N-(6-methoxy-methylquinolin-2-yl)cyclohexane-1,3-diamine;
N-(1H-indol-3-ylmethyl)-N′-(6-methoxy-4-methylquinolin-2-yl)cyclohexane-1,3-diamine;
N-(6-methoxy-4-methylquinolin-2-yl)-N′-(3-thienymethyl)cyclohexane-1,3-diamine;
N-(6-methoxy-4-methylquinolin-2-yl)-N′-[(1-methyl-1H-indol-3-yl)methyl]cyclohexane-1,3-diamine;
N-(1-benzofuran-2-ylmethyl)-N′-(6-methoxy-4-methylquinolin-2-yl)cyclohexane-1,3-diamine; N-(6-methoxy-4-methylquinolin-2-yl)-N′-(pyridin-2-ylmethyl)cyclohexane-1,3-diamine and
N-(4-methylquinolin-2-yl)-N′-(3-thienylmethyl)cyclohexane-1,3-diamine;
as well as pharmaceutically acceptable salts thereof.

15. A compound of formula I as claimed in any previous claim for use as a medicament.

16. A pharmaceutical formulation comprising a compound of formula I, as defined in any one of claims 1 to 14 and a pharmaceutically acceptable adjuvant, diluent or carrier.

17. Use of a compound of formula I, as defined in any one of claims 1 to 14 in the preparation of a medicament for the treatment or prophylaxis of conditions associated with obesity.

18. A method of treating obesity, psychiatric disorders, anxiety, anxio-depressive disorders, depression, bipolar disorder, ADHD, cognitive disorders, memory disorders, schizophrenia, epilepsy, and related conditions, and neurological disorders and pain related disorders, comprising administering a pharmacologically effective amount of a compound as claimed in any one of claims 1 to 14 to a patient in need thereof

19. A compound as defined in any one of claims 1 to 14 for use in the treatment of obesity.

20. A process for the preparation of compounds of formula I comprising reacting a compound of formula II 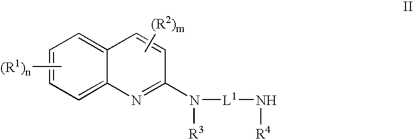
in which R¹, R², R³, R⁴, L¹, n and m are as previously defined with a compound of formula III
R⁵-L^{2′}=O III
in which R⁵ is as previously defined and L² represents a group which after reaction of compounds II and III gives L² on reduction, under reductive alkylation conditions.

21. Intermediates of formula II 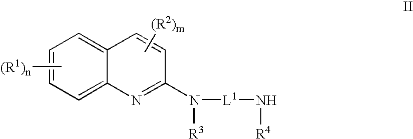
in which R¹, R², R³, R⁴, L¹, n and m are as defined in claim 1.
